(19) United States
(12) Patent Application Publication
(10) Pub. No.: US 2009/0083867 A1
(43) Pub. Date: Mar. 26, 2009

(54) Use of Antagonists of Cxcl13 or Cxcr5 for Treating Wounds of Fibrotic Diseases

(76) Inventors: Mark Williams James Ferguson, Manchester (GB); Sharon O'Kane, Manchester (GB); Neil French, Manchester (GB); Darren Hodgson, Manchester (GB); Nicholas Occleston, Manchester (GB)

Correspondence Address: NIXON & VANDERHYE, PC, 901 NORTH GLEBE ROAD, 11TH FLOOR, ARLINGTON, VA, 22203, US

(21) Appl. No.: 12/226,481

(22) PCT Filed: Apr. 20, 2007
(86) PCT No.: PCT/GB2007/001449

§ 371 Date: Oct. 20, 2008

(30) Foreign Application Priority Data
Apr. 20, 2006 (GB) 0607774.7

(51) Int. Cl. G01N 33/15; C07K 16/00; C07K 2/00; C07K 16/28; C07H 21/02; A01K 67/027

(52) U.S. Cl. 800/3; 530/387.1; 530/300; 530/388.22; 536/24.5; 536/23.1; 800/9

(57) ABSTRACT

The invention provides the use of an antagonist of CXCL 13 or CXCR5 activity in the preparation of a medicament for the prevention and/or inhibition of pathological scarring; as well as the use of such antagonists in the preparation of a medicament for the prevention and/or inhibition of fibrotic disorders; and the use of such antagonists in the preparation of a medicament for the prevention and/or treatment of a chronic wound. Medicaments or methods of the invention are particularly suitable for use in keloids or venous ulcers. Suitable antagonists of CXCL13 or CXCR5 activity may be any compound capable of inhibiting the CXCL13/CXCR5 signalling pathway. The invention also provides a model of aberrant wound healing, which may lead to pathological scar formation, or to chronic wound formation.

The present invention relates to the provision of medicaments and methods of treatment for use in the prevention and/or inhibition of pathological scarring. The invention also relates to medicaments and methods of treatment for use in the prevention and/or treatment of chronic wounds, and to medicaments and methods of treatment for use in the prevention and/or treatment of fibrotic disorders.

Post-natal wound healing in human tissues comprises a series of overlapping processes that ultimately result in the repair rather than regeneration of the damaged tissue (i.e. re-establishment of some, though not all, of the functions and structure of the original tissue). Wound healing involves a complex series of events, including hemostasis, inflammation, cell division, cell migration, and tissue remodelling. Both the rate of healing and the quality/functionality of scars produced after tissue repair are of major clinical importance. Perturbations of the normal healing process may lead to excessive pathological scar formation, or to the production of chronic wounds in which the healing process is significantly retarded.

Scarring represents the endpoint of the normal continuum of mammalian tissue repair, and has been defined as “the macroscopic disturbance of the normal structure and function of the skin architecture, resulting from the end-product of a healed wound”.

Scarring occurs after all types of surgery, penetrating trauma and burns. In the majority of cases the loss of function associated with scar formation may be viewed as an unwanted, but acceptable, outcome. However, in the case of pathological scarring the lesions produced as a result of the healing response may lead to serious deleterious effects.

Pathological scarring may be considered to embrace a number of forms of excessive scarring such as keloid scarring, hypertrophic scarring, and pterygium. Pathological scarring may incorporate an ongoing fibrotic component the resolution of which is delayed as compared to the resolution of fibrosis observed during normal healing of wounds (i.e. healing leading to non-pathological scarring).

Keloid scars (or keloids) constitute a notable example of pathological scarring, and are raised scars that spread beyond the margins of the original wound and invade the surrounding normal skin. Keloids continue to grow over time, do not regress spontaneously, and frequently recur following surgical excision. Keloid scars occur with equal frequency in men and women, mainly from ages 10 to 30, and can result from piercing, surgery, vaccination, tattoos, bites, blunt trauma and burns. A number of studies have suggested that there is an underlying genetic predisposition to keloid formation since keloid scars are more prevalent in dark skinned races.

Keloids appear as elevated scars that may typically be hyperpigmented or hypopigmented in relation to the surrounding tissue. Keloids may be characterised on the basis of their tendency to grow beyond the initial boundaries of a wound. At microscopic level, keloids may be characterised by the presence of large whorls of collagen, and the predominantly acellular nature of the interior of the lesion.

Current treatment regimes for keloids are empirical and include corticosteroid injections, cryotherapy, radiation therapy, silicone gel dressings and surgical excision, either alone or in combination. However, none of these methods are universally effective for the treatment of keloids. Due to the inability to effectively treat keloids, and their high incidence of recurrence, they are very problematic and cause great distress to the sufferer. Presently, prevention is paramount in keloid therapy and avoidance of non-essential cosmetic surgery is recommended in keloid-forming patients. However, it is generally accepted that the prevention or treatment of keloids remains an area of unmet medical need.

Hypertrophic scars are raised scars which may have an appearance very similar to keloid lesions. Unlike keloids, hypertrophic scars do not expand beyond the boundaries of the original injury and are not prone to recurrence after excision. Hypertrophic scars may frequently undergo contraction, and it is believed that the contractile nature of hypertrophic scars may be associated with the elevated numbers of myofibroblasts that are frequently reported within these types of scars. Hypertrophic scars may commonly arise as a result of burn or scald injuries, and are particularly common amongst children.

Pterygium is a hypertrophied outgrowth of the subconjunctival tissue to the border of the cornea or beyond. The outgrowth is typically triangular in shape, with the apex pointing towards the pupil. Pterygium may interfere with vision, and may require surgery to remove the hypertrophied tissue. Furthermore, the tissue may frequently re-grow after excision, in the same manner as keloid scars, thus requiring multiple rounds of surgery.

While pathological scarring may be considered to represent the damaging effects of an “over-exuberant” wound healing response, a decreased rate of wound healing also constitutes a perturbation of the normal healing response that may give rise to adverse effects.

The rate of healing is a prominent problem in the elderly where poor and non-healing wounds represent a significant health burden. For 2-3 percent of the U.S. population chronic wounds become a medical problem that can require specialized treatment and care. As opposed to acute wounds that typically heal in a matter of days or weeks, chronic wounds can persist for months or even years. Chronic wounds such as diabetic and venous ulcers are a considerable source of patient morbidity and expenditure for healthcare systems. In the UK alone, treatment of chronic wounds costs approximately £1 billion per year, a figure which is likely to expand due to an ageing population and marked increases in the number of diabetes sufferers (see Margolis et al, 2002; Kesavadev et al, 2003).

Chronic venous leg ulcers are a major health problem in the United Kingdom costing the National Health Service approximately ±400 million per year and affecting up to 1.5% of the elderly population. Of the more than 3.2 million people in the United States with lower extremity ulcers, 80 to 90 percent have ulcers secondary to venous insufficiency, which causes blood to accumulate in the lower legs. Venous abnormalities exist in 27 percent of the U.S. adult population, and 2 percent experience ulceration. Venous ulcers have recurrence rates of up to 80 percent.

The pathogenesis of venous ulceration is incompletely understood but is generally believed to be a direct consequence of venous insufficiency and sustained venous hypertension. Current methods for the healing of venous ulcers depend on leg elevation and compression. These methods are widely assumed to improve skin perfusion. Four-layer compression bandaging in specialist clinics achieves healing in 75% of venous ulcers within twelve weeks, although 10-15% of ulcers remain difficult to heal and many ulcers recur. An overall recurrence rate of 26% at one year, rising to 31% at eighteen months, has been reported in subjects with healed leg ulceration treated by compression. Thus, it can be seen that there remains a need for effective medicaments that may be used in the prevention and/or treatment of chronic wounds such as leg ulcers.

Although scarring may be defined as the structure produced on healing of a wound, similar disturbances of the extracellular matrix are also associated with a number of medical conditions known as fibrotic disorders. In these disorders excessive fibrosis leads to pathological derangement and malfunctioning of tissue (in a similar way to that seen in pathological scarring). Fibrotic disorders are characterised by the accumulation of fibrous tissue (predominately collagens) in an abnormal fashion within the damaged tissue. Accumulation of such fibrous tissues may result from a variety of disease processes which all lead to the same end result.

As has been mentioned above, pathological scars such as keloids also involve an ongoing fibrotic response, which does not naturally resolve itself in the manner seen during normal wound healing. This continuing fibrotic component may be associated with the propensity of pathological scars such as keloids and pterygium to grow beyond the boundaries of the original injury and to recur after surgical excision. The skilled person will appreciate that the ongoing fibrosis found in pathological scarring mirrors the fibrotic response observed in fibrotic disorders. It may be expected that the biological mechanisms responsible for fibrosis may be common between both pathological scarring and fibrotic disorders, and hence that methods and medicaments that may be used to prevent or reduce fibrosis in one condition may also be of utility in the other.

Fibrotic disorders are usually chronic. Examples of fibrotic disorders include cirrhosis of the liver, liver fibrosis, glomerulonephritis, pulmonary fibrosis, chronic obstructive pulmonary disease, scleroderma, myocardial fibrosis, fibrosis following myocardial infarction, central nervous system fibrosis following a stroke, neuro-degenerative disorders (e.g. Alzheimer's Disease, multiple sclerosis), proliferative vitreoretinopathy (PVR), arthritis, adhesions e.g. in the digestive tract, abdomen, pelvis, spine.

There remains a generally recognised need for medicaments and treatments that may be used to alleviate or cure fibrotic conditions such as those listed above.

Whilst the above considerations mainly apply to conditions, disorders or diseases of man, it will be appreciated that aberrant wound healing (for instance wound healing leading to pathological scarring) and fibrotic disorders can also be problematic in other animals, particularly veterinary or domestic animals (e.g. horses, cattle, dogs, cats etc). For instance, abdominal wounds or adhesions are major reasons for having to put down horses (particularly race horses), as are tendon and ligament damage leading to excessive scarring or fibrosis similar to that observed in human keloids.

The chemokines are a large superfamily of cytokines originally characterised as controllers of the movement of immune cells. Chemokines have been functionally classified as either inducible chemokines, which have an inflammatory function, or as constitutive chemokines which function as “homing” molecules related to normal trafficking of immune cells. All members of the superfamily share an overall structure comprising three β-sheets and a carboxy-terminal helix. Chemokines may be structurally classified on the basis of a cysteine motif near their N-terminus; adjacent cysteines for the “CC” chemokines and “CXC” for those with an intervening amino acid.

Known chemokine receptors are class A seven-transmembrane-spanning G-protein coupled receptors (GPCRs). In contrast to many other cytokine receptors the GPCRs have proven pharmaceutically tractable targets with many small-molecule antagonists marketed as drugs

CXCL13 (also known as BCA-1, BLC, SCYb13, BLR1L, Angie, ANGIE2) is a ‘homing’ chemokine, expression of which is normally restricted to B-cell follicles. CXCR5 (also known as BLR1, MDR15) is the known receptor for CXCL13 and is mainly expressed on mature B-lymphocytes and Burkitt's lymphoma cells. CXCL13 is known to attract naive B-cells and certain activated and memory T-cells.

Mice with impaired CXCL13 or CXCR5 lack inguinal lymph nodes, have profoundly altered germinal centres in the spleen and have few/abnormal Peyer's patches (Ansel et al, 2000). Transgenic expression of CXCL13 induces the ectopic generation of lymph node like structures and expression of CXCL13 has been linked with the inappropriate formation of ectopic lymphoid follicles in several, predominantly autoimmune, disease states.

Human CXCL13 cDNA encodes a protein of 109 amino acids with a 22 amino-acid leader sequence. CXCL13 human and mouse proteins differ by several amino acids (64% identity). Human, rat and mouse CXCR5 proteins have high identity (74% human-mouse; 72% human-rat).

The amino acid sequence of human CXCL13 (both with and without leader seqeuence), as well as the sequence of cDNA encoding CXCL13 are provided in the Sequence Information section. The Sequence Information section also provides the amino acid sequences of two isoforms of CXCR5, the main form (isoform 1 shown in Sequence ID No. 3) and a shorter splice variant (isoform 2 shown in Sequence ID No. 4) as well as cDNA molecules encoding these forms of the receptor.

It is an aim of the present invention to obviate or mitigate at least some of the disadvantages of the prior art. In particular it is an aim of certain embodiments of the invention to provide new medicaments and methods of treatment for use in the prevention and/or inhibition of pathological scarring. It is also an aim of certain embodiments of the invention to provide new medicaments and methods of treatment for use in the prevention and/or treatment of chronic wounds. It is also an aim of certain embodiments of the invention to provide new medicaments and methods of treatment for use in the prevention and/or treatment of fibrotic disorders.

In a first aspect of the present invention there is provided the use of an antagonist of CXCL13 or CXCR5 activity in the preparation of a medicament for the prevention and/or inhibition of pathological scarring.

The present invention is based on the inventors' surprising finding that antagonism of CXCL13 or CXCR5 activity is capable of preventing or reducing pathological scar formation. It will be appreciated that, since CXCL13 and CXCR5 constitute a specific ligand/receptor signalling pair, therapeutic effects may be achieved through the antagonism of either (or both) of these molecules. There is nothing in the prior art that would indicate that the formation of pathological scars may be prevented or reduced by antagonism of CXCL13 or CXCR5. The prevention and/or treatment of disfiguring scars, such as pathological scars, is an area of great clinical, scientific and commercial interest.

The inventors have found that antagonism of CXCL13 or CXCR5 activity is particularly effective in the prevention and/or treatment of pathological scars selected from the group consisting of keloid scars, hypertrophic scars, and pterygium. Therapeutic antagonism of CXCL13 or CXCR5 activity is particularly preferred for the prevention and/or treatment of keloid scars. The inventors believe that the methods and medicaments of the present invention may be particularly suitable for the treatment of wounds that are predisposed to form keloids.

The inventors have found that expression of CXCL13 is greatly increased in acute wounds that are destined to develop pathological scarring. For example, acute wounds that will eventually give rise to keloid scars exhibit levels of expression of CXCL13 that are up to 20-fold higher than expression found in ethnically matched controls. There is currently a dearth of treatments available for the prevention, treatment or inhibition of pathological scaring, and the present invention thus provides an important addition to the state of the art as it relates to this problematic area of wound healing. The development of new medicaments manufactured in accordance with the first aspect of the invention raises the prospect of new therapies that may be used in the prevention treatment or inhibition of pathological scarring. Medicaments manufactured in accordance with the first aspect of the present invention may be particularly useful in the treatment of keloids, or other forms of pathological scarring prone to spontaneous recurrence. Accordingly, the invention provides the use of an antagonist of CXCL13 or CXCR5 activity in the preparation of a medicament for the prevention, treatment or inhibition of keloid scars.

In addition to the surprising uses outlined above, the inventors have also found that the antagonism of CXCL13 or CXCR5 may be useful to prevent the formation of chronic wounds, or to promote the healing of existing chronic wounds. Expression of mRNA encoding CXCL13 is significantly increased (up to 58-fold) at the margins of chronic venous ulcers as compared to in patient-matched acute wounds. Without wishing to be bound by any hypothesis, the inventors believe that the resultant increase in CXCL13 activity contributes to the development and/or maintenance of chronic wounds. Accordingly, in a second aspect of the invention there is provided the use of an antagonist of CXCL13 or CXCR5 activity in the preparation of a medicament for the prevention and/or treatment of chronic wounds.

Chronic wounds that may be prevented and/or treated through the antagonism of CXCL13 or CXCR5 activity include venous ulcers particularly venous ulcers of the lower extremities such as the legs and feet), ischemic wounds, decubitus ulcers (pressure sores), wounds infected with microorganisms, and wounds in patients with impaired circulation or venous stasis, or diabetic ulcers especially of the foot. Venous ulcers constitute a preferred subset of chronic wounds that may be prevented or treated in accordance with the present invention.

Medicaments in accordance with the second aspect of the invention may be used in the prevention of chronic wounds in subjects at risk of developing such wounds. A risk of chronic wound development may be indicated by the general health status of a patient. For example, aged patients or diabetic patients may be considered generally to be at elevated risk of developing chronic wounds. Alternatively, or additionally, increased risk of chronic wound development may be indicated by predisposing symptoms or features such as the presence of lipodermatosclerotic skin, or the presence of the factor V Leiden mutation (Maessen-Visch et al, 1999). Alternatively, or additionally, certain types of wound, such as pre-tibial lacerations, may be considered to be associated with a predisposition to chronic wound development.

When medicaments manufactured in accordance with the second aspect of the invention are to be used prophylactically they may preferably be administered at the earliest possible time when risk of chronic wound formation has been recognised. Most preferably medicaments manufactured in accordance with the second aspect of the invention may be administered to patients susceptible to chronic wound formation prior to the manifestation of symptoms. It will be appreciated that medicaments manufactured in accordance with the second aspect of the invention are particularly suitable for use in the prevention of recurrence of chronic wounds.

Although they do not wish to be bound by any hypothesis, the inventors believe that antagonists of CXCL13 or CXCR5 activity promote the healing of chronic wounds by increasing the rate of healing exhibited by wounds subject to such treatment. Accordingly, the skilled person will recognise that the present invention encompasses the use of antagonists of CXCL13 or CXCR5 activity in the preparation of a medicament for accelerating the healing of chronic wounds. The skilled person will appreciate that the inventors' findings are particularly surprising, since they contradict the previously held belief that agonists of certain chemokines (rather than antagonists, as disclosed in the present invention) may have use in the acceleration of wound healing.

For the purposes of the present invention, a chronic wound may be defined as any wound that does not show any healing tendency within eight weeks of formation when subject to appropriate (conventional) therapeutic treatment.

Chronic wounds such as venous ulcers are considered by some to be a secondary feature of underlying skin disorders such as lipodermatosclerosis. It will therefore be appreciated that the current invention provides medicament, and methods of treatment suitable for the treatment of lipodermatosclerosis to reduce the incidence of chronic wounds (such as venous ulcers).

In a third aspect of the present invention there is provided the use of an antagonist of CXCL13 or CXCR5 activity in the preparation of a medicament for the prevention, treatment, or inhibition of a fibrotic disorder. Without wishing to limit the scope of the present invention, it will be appreciated that medicaments manufactured in accordance with this third aspect of the invention may be used in the prevention, treatment or inhibition of fibrosis associated with fibrotic disorders selected from the group consisting of: cirrhosis of the liver, liver fibrosis, glomerulonephritis, pulmonary fibrosis, lung fibrosis, scleroderma, skin fibrosis, muscle fibrosis, radiation fibrosis, kidney fibrosis, uterine fibrosis, adhesions such as those occurring in the abdomen, pelvis, spine, chronic obstructive pulmonary disease, scleroderma, myocardial fibrosis, fibrosis following myocardial infarction, central nervous system fibrosis following stroke, fibrosis associated with neuro-degenerative disorders (e.g. Alzheimer's Disease or multiple sclerosis) and fibrosis associated with proliferative vitreoretinopathy (PVR). It may be preferred that the medicaments or methods of the invention be used in the prevention, treatment or inhibition of conditions other than those of the respiratory system.

For the purposes of the present specification, references to “antagonists of CXCL13 or CXCR5 activity” are to be understood to encompass any substance, or mixture of substances, capable of antagonising the biological activity of CXCL13 that may be elicited through the CXCR5 receptor. This term is intended to encompass substances or mixtures of substances that antagonise CXCL13 activity and also antagonise CXCR5 activity; substances or mixtures of substances that antagonise CXCL13 activity but do not directly antagonise CXCR5 activity (for example by binding CXCL13 and thereby preventing it binding to, and signalling through, CXCR5); and substances or mixtures of substances that antagonise CXCR5 activity but do not directly antagonise CXCL13 activity (for example by binding to CXCR5, but not CXCL13, and thereby preventing signalling that may otherwise occur on binding of CXCL13 to the receptor). References to antagonists of CXCL13 or CXCR5 activity may be taken to encompass inhibitors of the CXCL13/CXCR5 signalling pathway.

Medicaments manufactured in accordance with the present invention may preferably be for use in human patients.

Generally, antagonists suitable for use in accordance with the present invention should be taken to encompass simple chemical organic or inorganic compounds capable of antagonising CXCL13 or CXCR5 activity; and also to include proteins, peptides, nucleic acids, sugars and antibodies or antibody derivatives having suitable antagonistic effects. Suitable interactions involved in antagonism of the CXCL13/CXCR5 signalling pathway may be covalent or non-covalent, and may be discovered by screening technologies well established in the pharmaceutical industry.

Antagonists suitable for use in the methods and medicaments of the present invention include those capable of antagonising the activity of wild-type CXCL13 or CXCR5, as well as those capable of antagonising variant forms of CXCL13 or CXCR5. For instance, suitable antagonists may include those capable of antagonising naturally occurring variants of CXCL13 or CXCR5, such as the short splice variant of CXCR5 set out in Sequence ID No. 4.

Suitable antagonists of CXCL13 activity or CXCR5 activity may include agents capable of decreasing the level of biologically active CXCL13 or CXCR5 present at a site of interest. For example, suitable antagonists may decrease the level of CXCL13 or CXCR5 mRNA or protein. Illustrative examples of antagonists of this type include antisense oligonucleotides that neutralise mRNA encoding CXCL13 or CXCR5, ribozymes capable of targeting and degrading mRNA encoding CXCL13 or CXCR5, or aptamers capable of binding to mRNA encoding CXCL13 or CXCR5. In each case the inactivation or degradation of mRNA will lead to a decrease in expression of the encoded protein.

The amino acid sequences of CXCL13 and CXCR5 are shown as Sequence ID Nos. 1, 2, 3 and 4 respectively, and the sequences of cDNA molecules encoding CXCL13 or CXCR5 are shown as Sequence ID Nos. 5, 6 and 7 respectively. The skilled person will thus be readily able to devise suitable sequences of oligonucleotides or ribozymes complementary to mRNA encoding CXCL13- or CXCR5. Furthermore, the skilled person may use the information provided in the specification to allow the development of aptamers capable of binding to such mRNAs encoding CXCL13 or CXCR5.

A further class of nucleic acids suitable for use in medicaments and methods of treatment according to the invention comprise double-stranded or hairpin short-interfering RNAs (or RNA derivatives) specific to mRNA encoding CXCL13 or CXCR5. Suitable interfering RNAs or derivatives thereof may be chemically synthesised in vitro or produced in vitro or in vivo from suitable vectors (Montgomery, 2004; Schutze, 2004).

In general, oligonucleotides are degraded relatively rapidly in vivo. It may therefore be preferred to modify nucleic acid inhibitors to be used in the medicaments and methods of the invention in order to stabilise them against degradation. Suitable methods may be based on those disclosed in Kurreck, 2003; or those in WO 97/29116.

For the purposes of the present invention, antagonists of CXCL13 activity or CXCR5 activity suitable for use in the medicaments or methods of the invention include compounds (for example other than nucleic acids of the type considered above) that are capable of preventing or reducing the induction of CXCL13 or CXCR5. One example of such a compound is LTβR-Ig, which is a fusion protein combining parts of the lymphotoxin β receptor (LTβR) and immunoglobulin IgG1. LTβR-Ig acts as a “decoy” receptor, competing with LTβR (a normal function of which is to induce CXCL13), and thereby reducing induction of CXCL13. LTβR-Ig represents a preferred antagonist of CXCL13 activity or CXCR5 activity that may be used in the methods or medicaments of the invention. It will be appreciated that LTβR-Ig may be used as a “template” for the generation of further antagonists of CXCL13 activity, or CXCR5 activity, suitable for use in the medicaments or methods of the invention. These may be produced using methods previously described for the generation of LTβR-Ig, and may preferably utilise human LTβR in combination with proteins such as human immunoglobulins.

Other suitable antagonists of CXCL13 activity or CXCR5 activity suitable for use in accordance with the present invention are those capable of directly competing with CXCL13 for binding to CXCR5 receptor molecules, or those capable of directly competing with CXCR5 for binding to CXCL13 ligand. Ligand-binding portions of the CXCR5 receptor that lack the domains associated with intracellular signalling constitute an example of the latter class of antagonists.

Another antagonist of CXCL13 activity or CXCR5 activity that may be used in the medicaments or methods of the invention is the 44 kDa protein encoded by the M3 gene of the murine gammaherpesvirus 68 (MHV-68). This protein has been shown to block the activity of many chemokines, including CXCL13, but has not previously been suggested to have utility in the prevention and/or treatment of pathological scarring, or of fibrotic disorders or chronic wounds.

Suitable antagonists of CXCL13 activity or CXCR5 activity for use in accordance with the present invention include those antagonists that interfere with binding of CXCL13 to CXCR5 through blocking either the receptor-binding portion of CXCL13 or the ligand-binding site of CXCR5. Examples of antagonists in accordance with this embodiment of the invention include antibodies, aptamers or peptides that are able to block the function of CXCL13 or CXCR5.

Antibody antagonists suitable for use in accordance with the present invention include neutralising antibodies (or antibody derivatives) capable of binding to CXCL13 or to a receptor for CXCL13 such as CXCR5. Antibodies suitable for use in medicaments or methods of treatment of the invention may bind to CXCL13 or to CXCR5 such that the biological activity of these molecules is antagonised. It will be appreciated that suitable antibodies may preferably exhibit specific binding to CXCL13 or CXCR5. Antibodies that may be used in the medicaments or methods of treatment of the invention include monoclonal antibodies and polyclonal antibodies, as well as fragments of such antibodies, including, but not limited to, Fab or F(ab′)hd 2, and Fv fragments.

For instance, two function-blocking antibodies that may be used to antagonise CXCL13 or CXCR5 activity are commercially available from R&D Systems. MAB801 is a monoclonal antibody that binds specifically to human CXCL13. MAB190 is a monoclonal antibody that binds specifically to human CXCR5. That both MAB801 and MAB190 are able to antagonise the activities of CXCL13 and CXCR5 is illustrated by the fact that both of these agents may be used (independently) to inhibit chemotactic activity mediated through CXCL13/CXCR5 signalling. Thus, MAB801 or MAB190 may be used, either independently or in combination, in the medicaments or methods of the invention, and such uses represent preferred embodiments of the invention.

Methods suitable for the generation and/or identification of antibodies capable of binding specifically to a given target (such as CXCL13 or CXCR5) are well known to those skilled in the art. In general suitable antibodies may be generated by the use of the isolated target as an immunogen. This immunogen is administered to a mammalian organism, such as, but not limited to, a rat, rabbit, goat or mouse and antibodies elicited as part of the immune response. Generally antibodies will be used in the context of the methods and kits of the invention to bind to protein products of gene expression. Suitable immunogens may include the full-length protein to be investigated, or an antigenic peptide fragment thereof.

Monoclonal antibodies can be produced by hybridomas, immortalized cell lines capable of secreting a specific monoclonal antibody. The immortalized cell lines can be created in vitro by fusing two different cell types, usually lymphocytes, one of which is a tumour cell.

As indicated above, aptamers represent a further class of antagonists suitable for use in the medicaments or methods of treatment of the invention. Aptamers are nucleic acid molecules that assume a specific, sequence-dependent shape and bind to specific target ligands based on a lock-and-key fit between the aptamer and ligand. Typically, aptamers may comprise either single- or double-stranded DNA molecules (ssDNA or dsDNA) or single-stranded RNA molecules (ssRNA).

Aptamers may be used to bind both nucleic acid and non-nucleic acid targets. Accordingly aptamers are suitable for antagonism of CXCL13 and/or CXCR5 either through binding to the expressed protein, or through binding to nucleic acids encoding the relevant proteins.

Suitable aptamers may be selected from random sequence pools, from which specific aptamers may be identified which bind to the selected target molecules with high affinity. Methods for the production and selection of aptamers having desired specificity are well known to those skilled in the art, and include the SELEX (systematic evolution of ligands by exponential enrichment) process. Briefly, large libraries of oligonucleotides are produced, allowing the isolation of large amounts of functional nucleic acids by an iterative process of in vitro selection and subsequent amplification through polymerase chain reaction.

The use of aptamers in medicaments or methods of treatment of the invention may be advantageous, since aptamers are relatively stable, and thus have long shelf lives. Aptamers suitable for use in the medicaments and/or methods of the invention may preferably be stabilized by chemical modifications (for example 2′—NH₂ and 2′-F modifications).

Antagonists suitable for use in the medicaments and methods of the invention also include receptor-blocking agent such as function neutralising peptides. Such peptides may include mimics of the ligand-binding region of the receptor (which are able to compete with the naturally occurring receptor, but do not give rise to CXCL13 activity on binding to the peptide mimic) and/or naturally occurring binding proteins or their derivatives, as well as soluble forms of the ligand-binding domain of the CXCR5 receptor.

Suitable antagonists may include agents capable of indirectly reducing incidences of binding between CXCL13 and the CXCR5 receptor, for example through sequestration of CXCL13 or CXCR5.

A further class of antagonists of CXCL13 or CXCR5 activity suitable for use in accordance with the present invention comprises substances or mixtures of substances that are capable of indirectly modulating signalling events elicited by the binding of CXCL13 to CXCR5.

The skilled person will appreciate that antagonism of intracellular signalling arising on binding of CXCL13 to CXCR5 represents a suitable means by which CXCL13 or CXCR5 activity may be antagonised. The discovery and optimisation of antagonists of GPCRs such as CXCR5 via screening and molecular modelling is well-established, and examples of small molecule antagonists of the CXCR family antagonists are known to those skilled in the art.

By way of example, CXCR5 is known to signal through the G proteins G_{α}_{₁} and G_{α}_{₁}. The regulator of G protein signalling (RGS) proteins RGS1 and RGS13 are both known to impair signalling through G proteins such as G_{α}_{₁} and G_{α}_{₁}. Accordingly, the skilled person will appreciate that RGS1 or RGS13 (or compounds capable of increasing expression or activity of these proteins) may be used to effectively antagonise CXCL13 or CXCR5 activity as required by the invention.

Alternatively, or additionally, intracellular signalling from CXCR5 may be disrupted by inactivating G proteins associated with this receptor, such as G_{α}_{₁}. Suitable compounds capable of bringing about such inactivation include the bacterial pertussis toxin (which acts to “lock” the G_{α}_{₁}, protein in its inactive state) and the peptide G protein antagonist G9541. Both of these agents are commercially available from Sigma.

In a preferred embodiment, medicaments manufactured in accordance with the first aspect of the invention may be used in the prevention, inhibition or treatment of keloid scars. Medicaments in accordance with this embodiment may be used to treat existing keloid scars, or to prevent the formation of new keloid scars in those predisposed to keloid formation. An individual's risk of keloid formation (which may be indicative of any predisposition to keloid formation) may be assessed with reference to presence of existing keloids at other body sites of the patient, or other indicators such as race, age, hormonal status and/or family history. Medicaments to be used for the prevention of keloids may preferably be administered prophylactically, prior to the manifestation of symptoms.

Medicaments manufactured in accordance with this preferred embodiment of the first aspect of the invention may be useful in the prevention of keloid formation in susceptible individuals, improvement of existing keloids, improving the quality of life of patients with keloids, and in providing an adjunct to surgery in the prevention of recurrence of keloids. Suitable antagonists for use in the manufacture of medicaments for the prevention and/or inhibition of keloids may be ones that are capable of bringing about at least one of the following favourable outcomes:
i) Reduction of recurrence of keloids at treated wound sites.
ii) Extension of the time elapsed before first recurrence of a keloid at a treated site.
iii) Reduction of the size and/or volume of a keloid in relation to the original volume of the lesion, or to matched untreated keloids.
iv) Reduction of and/or elimination of the spread of the keloid in relation to the size of the keloid at treatment.
v) Increased patient satisfaction on treatment of keloids.
vi) Degree of movement permitted by treated keloids as opposed to untreated keloids (or in relation to the same keloid prior to treatment).
vii) Scar perception in relation to original keloid; this may be assessed by investigator and/or patient (in real time) and/or lay panel (assessment of images).

Medicaments manufactured in accordance with the second aspect of the invention may prevent formation of chronic wounds in susceptible individuals, improve healing in patients with chronic wounds, improved quality of life in patients with chronic wounds, reduce health care provider costs associated with the treatment of chronic wounds and the prevention of recurrence of chronic wounds.

Suitable antagonists for use in the manufacture of medicaments in accordance with the second aspect of the invention include those able to give rise to at least one of the following beneficial outcomes.
i) Prevention and reduction of recurrence of chronic wounds. This may be assessed with reference to the number of susceptible patients developing a chronic wound of a specified severity during a fixed period of time.
ii) Improvement of quality of life of chronic wound sufferers. This may be assessed by means of a questionnaire covering major issues of concern for sufferers of chronic wounds e.g. pain.
iii) Improved healing of chronic wounds. This may be assessed with reference to the number of treated chronic wounds that demonstrate a significant reduction in wound size; and/or completely heal within a fixed period of time and/or; exhibit a decrease in the average time taken to achieve complete healing; and/or exhibit a decrease in the time taken for the wound to heal to allow routine closure by another technique e.g. skin grafting.
iv) Decrease in the cost of health care provision to sufferers of chronic wounds.
v) Decrease in the time taken for the ulcer to heal to a degree associated with a significant reduction in health care costs or improved quality of life.

Typically the size and shape of chronic wounds may be periodically monitored by tracing onto an acetate sheet or by photographic record.

The inventors have found that the location in which CXCL13 or CXCR5 activity occurs in wounds destined to become chronic wounds (such as venous ulcers) or pathological scars (such as keloids) is also important in optimising the therapeutic use of inhibitors of these factors. CXCL13 and CXCR5 activity is most elevated at the margins of wounds that subsequently develop into pathological scars, and also in the margins of chronic wounds. Accordingly it may be preferred that medicaments in accordance with the present invention be administered at the edges of wounds to be treated. Suitable forms for such administration may include topical formulations suitable for application at the wound margins and injectable solutions suitable to be injected into the margins of wounds.

The “prevention” or “inhibition” of pathological scarring in the context of the present invention should be taken to encompass any clinically significant reduction of an existing pathological scar, or of a pathological scar formed on treatment in accordance with the methods and/or medicaments of the present invention.

Both pathological scarring and fibrosis may have detrimental effects in the eye. As described above, pterygium is a form of pathological scarring consisting of a raised, wedge-shaped fibrotic growth of the conjunctiva. It is common among those who spend a lot of time in the sun and/or in dusty conditions. For some, the growth remains dormant; however, in other cases it grows over the central cornea and affects the vision. If the pterygium invades the central cornea, it is removed surgically, although growth may recur after surgical excision. In determining the likelihood of incidences of pathological scarring in the eye surgeons may investigate a patient's existing scars at other body sites. The presence of keloid or excessive scarring may be considered as an indicator of increased likelihood of pathological scarring in the eye.

The inventors also believe that medicaments and methods of treatment in accordance with the present invention may be used in the prevention and/or inhibition of fibrosis in the eye, such as fibrosis associated with proliferative vitreoretinopathy.

Fibrosis in the heart or cardiovascular system (e.g. following myocardial infarction or restenosis) can give rise to abnormal cardiac function. The inventors believe that medicaments and methods of treatment in accordance with the present invention may be used in the prevention and/or inhibition of fibrosis in the heart or cardiovascular system.

Fibrosis of the reproductive tract, such as uterine fibrosis or fallopian tube adhesions, can lead to infertility. The inventors believe that medicaments and methods of treatment in accordance with the present invention may be used in the prevention and/or inhibition of fibrosis in the reproductive tract.

Antagonists may be administered at any concentration suitable to achieve a therapeutically effective decrease in CXCL13 or CXCR5 activity. Methods by which suitable concentrations may be determined are well known to those skilled in the art, and are considered further below. By way of example, antagonists, such as neutralising antibodies, may preferably be administered to achieve a localised concentration of the antagonist (at the site where they are to have their therapeutically effective antagonistic activity) of between 0.001 and 250 μg/ml, preferably between 0.01 and 75 μg/ml, or most preferably between 0.1 and 50 μg/ml. For instance, the CXCL13 neutralising antibody MAB801 referred to above may achieve therapeutically effective antagonism at a local concentration of between 1 and 100 μg/ml, preferably between 10 and 50 μg/ml, more preferably between 15 and 45 μg/ml, and most preferably at about 30 μg/ml. By way of comparison the CXCR5 neutralising antibody MAB190 may achieve therapeutically effective antagonism at a local concentration of between 0.01 and 5 μg/ml, preferably between 0.1 and 1 μg/ml, more preferably between 0.3 and 0.9 μg/ml, and most preferably at about 0.6 μg/ml.

Antagonists may be formulated in non-toxic, inert, pharmaceutically acceptable carriers such that the antagonist will have biological activity upon release from the carrier. Furthermore, in some instances suitable antagonists may be synthesised in situ using a suitable vector (such as a plasmid, or viral vector). Expression from such vectors may be constitutive or cell-type specifically inducible or inducible upon activation with a further compound. Formulations may be delivered by known routes of administration including but not limited to topical creams or gels; transdermal patch or bandage; transmucosal spray or aerosol; wound irrigation solutions; injectable intravenous or lavage formulations; or orally administered liquid or pills.

Therapeutic formulations of suitable antagonists for localised parenteral administration (e.g. intradermal, intramuscular and subcutaneous) and systemic parenteral administration (e.g. intravenous and intra-arterial) may be prepared by mixing suitable antagonists (having the desired degree of purity) with optional physiologically acceptable carriers, excipients or stabilizers in the form of; lyophilised and non-lyophilised powder formulations for reconstitution prior to use, non-aqueous and aqueous solutions, and semi-solid formulations. Acceptable carriers, including excipients, are non-toxic to recipients at the dosages and concentrations employed, and include, but are not limited to, buffers such as phosphates, citrates, and other organic acids; antioxidants including ascorbic acid and methionine; tonicity modifiers such as sodium chloride, glucose, glycerol, and alike; preservatives such as octadecyldimethylbenzyl ammonium chloride; hexamethonium chloride; benzalkonium chloride, benzethonium chloride; phenol, butyl or benzyl alcohol; alkyl parabens such as methyl and/or propyl and/or butyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; other sugars such as sucrose, mannitol, maltose, trehalose or sorbitol; salt-forming counter-ions such as sodium; metal complexes (e.g., Zn-protein complexes); anionic surfactants such as fatty acid soaps, acyl sulfates, or acyl sulfosuccinates; cationic surfactants, such as alkyl primary, secondary, tertiary, or quaternary amines; non-ionic surfactants, for example, sorbitan esters or polyethoxylated esters of acyl acids, copolymers of polyethylene oxide and polypropylene oxide.

A medicament of the present invention comprising a sterile solution for parenteral administration, in addition to a suitable antagonist, may include the following: 0.01 to 0.1 M phosphate buffer, sodium chloride up to 0.9% w/v (to achieve isotonicity with blood, 290-300 mOsm/L) and 1 to 10 w/v % maltose (or alternatively another sugar). A lyophilized (freeze-dried) powder ‘cake’ of the above solution could be prepared. Medicaments in accordance with the invention may be presented in the form of a vial, an ampoule, or a pre-filled syringe of, either; a sterile solution, a sterile lyophilized (freeze-dried) powder for reconstitution, a sterile suspension or any other pharmaceutically acceptable form of presentation suited to localised parenteral drug delivery.

Therapeutic formulations of medicaments in accordance with the invention for topical administration (e.g. transdermal/cutaneous, ocular, otic, nasal, pharyngeal, buccal, rectal, vaginal, urethral) may be prepared by mixing suitable antagonists having the desired degree of purity with optional physiologically acceptable carriers, excipients or stabilisers in the form of lyophilised or non-lyophilised powder formulations, non-aqueous or aqueous solutions, non-aqueous or aqueous dispersions/suspensions, including emulsions and semi-solid formulations. Acceptable carriers, including excipients, are non-toxic to recipients at the dosages and concentrations employed, and include, but are not limited to, purified water, saline, phosphate-buffered saline (PBS), Ringer's solution, Ringer's-lactate solution, dextrose solutions, dextrose/saline solution, hydro-alcoholic solutions, glucose, sucrose, dextran, mannose, mannitol, maltose, sorbitol, polyethylene glycol (PEG), propylene glycol (PG), phosphates, acetates, gelatin, collagens, Carbopol 934™ (BF Goodrich Corp.), vegetable and synthetic oils and waxes, anionic surfactants such as fatty acid soaps, acyl sulfates, or acyl sulfosuccinates; cationic surfactants, such as alkyl primary, secondary, tertiary, or quaternary amines; non-ionic surfactants, for example, sorbitan esters or polyethoxylated esters of acyl acids, copolymers of polyethylene oxide and polypropylene oxide, and the like. Medicaments in accordance with the invention may additionally include suitable preservatives, stabilisers, antioxidants, anti-microbials and buffering agents, for example, methyl and/or propyl and/or butyl parabens, butylated hydroxy anisole (BHA), butylated hydroxy toluene (BHT), citric acid, ascorbic acid, and the like. Emulsion, cream or ointment bases useful in formulation of medicaments in accordance with the invention may include aqueous-based creams and emulsions (oil-in-water), oil-based creams and emulsions (water-in-oil), ointments (emulsifying and non-emulsifying hydrocarbon), gels, hydrogels, and the like. Other formulations suitable for topical delivery may include aerosols, bandages, and other wound dressings. For example suitable antagonists may be incorporated in dressings such as: bandages; film dressings; sponge dressings; or the like that may be applied to wounds. For example, it is suggested that sponge dressings incorporating suitable antagonists may be used in combination with negative pressure therapies to prevent or treat chronic wounds. Alternatively medicaments in accordance with the present invention may incorporate or encapsulate suitable antagonists in a suitable polymer matrix or membrane, thus providing a sustained-release delivery device suitable for placement on, or implantation near, a site to be treated.

A pharmaceutical formulation of a medicament in accordance with the invention in the form of a semi-solid hydrogel formulation for topical administration, in addition to suitable antagonists, may include the following: 0.1% w/v to 2.0% w/v hydroxy cellulose, 0.1% w/v to 1.0% w/v Carbopol 934™ (BF Goodrich Corp.), 10 to 20% w/v propylene glycol, 0.005% w/v to 0.020% w/v methyl paraben, and 0.005% w/v to 0.020% w/v propyl paraben, sodium hydroxide or hydrochloric acid q.s. ad pH 4-10, and purified water, q.s. ad 100% w/v. A medicament in accordance with the invention in keeping with this example may be presented in the form of a bottle, a jar, a tube, a spray, of, either; a sterile solution; a sterile lyophilized (freeze-dried) or non-lyophilised powder for reconstitution, a sterile dispersion/suspension, a sterile semi-solid, or any other pharmaceutically acceptable form of presentation suited to topical drug delivery.

Antagonists of CXCL13 or CXCR5 activity may also be administered systemically. In one embodiment a pharmaceutical vehicle for systemic administration of a suitable antagonist may be a liquid and a suitable pharmaceutical composition would be in the form of a solution. In another embodiment, the pharmaceutically acceptable vehicle is a solid and a composition for systemic administration of a suitable antagonist is in the form of a powder or tablet. In a further embodiment a suitable antagonist may be formulated as a part of a pharmaceutically acceptable transdermal patch.

A solid vehicle can include one or more substances which may also act as flavouring agents, lubricants, solubilizers, suspending agents, fillers, glidants, compression aids, binders or tablet-disintegrating agents; it can also be an encapsulating material. In powders, the vehicle is a finely divided solid which is in admixture with the finely divided antagonist. Such powders may preferably be provided in capsule form (i.e. within a degradable capsule coating). In tablets, the antagonist is mixed with a vehicle having the necessary compression properties in suitable proportions and compacted in the shape and size desired. The powders and tablets preferably contain up to 99% of the selected antagonist. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

A still further formulation suitable for systemic or topical administration of suitable antagonists may be in the form of an inhalable composition. Such a composition may utilise a solid formulation (such as a finely divided powder) or a suitable solution (administered in the form of a mist). It will be appreciated that inhalation may be used to enable systemic administration of suitable antagonists, as well as to provide topical administration of antagonists to sites of fibrosis occurring in the lungs or other respiratory tissues.

It may be preferred to utilise dosage forms suitable for systemic administration (such as the tablet, capsule or inhalation forms discussed above) in the prevention of chronic wound formation, since this method may help generate a “protective” therapeutically effective amount of an inhibitor at sites where, although no injury has yet occurred, a chronic wound may subsequently form. Such systemic formulations may be particularly effective for administration to those exhibiting conditions, such as lipodermatosclerosis, that indicate an increased likelihood of later chronic wound formation. It will also be appreciated that dosage forms giving rise to a systemic effect may be particularly useful in the prevention, treatment, inhibition or reduction of systemic fibrotic disorders, or fibrotic disorders occurring at inaccessible body sites (such as internal organs or tissues).

A number of models for chronic wounds currently exist; however, none of these models are widely accepted to mimic a human chronic wound by those skilled in the art. The inventors believe that experimental animals with high levels of CXCL13 in their skin (for example as a result of administration of CXCL13, or preferably as a result of over-expression of CXCL13) may provide a suitable animal model for chronic wounds such as venous ulcers.

No accepted animal models for keloid scarring exist. The inventors believe that animals with high levels of CXCL13 in their skin may also constitute a clinically relevant model for keloid scarring. As above, high levels of dermal CXCL13 may be achieved by the administration of CXCL13, or preferably through the over-expression of CXCL13.

Accordingly, in a further aspect the present invention provides the use of a transgenic animal that over-expresses CXCL13 in a model of aberrant wound healing.

The invention also provides a method of screening a test compound for use in the prevention, treatment or inhibition of aberrant wound healing, the method comprising administering the test compound to a dermal wound site of a transgenic animal that over-expresses CXCL13. The wound treated with the test compound (the “test wound”) may then be compared to a reference wound in order to assess whether or not the test wound has undergone aberrant wound healing.

A suitable reference wound may be an untreated wound of a transgenic animal that over-expresses CXCL13, in which case the test compound may be suitable for use in the prevention, treatment and/or inhibition of aberrant wound healing if the test wound is more similar to a normal healing wound than is the reference wound. Alternatively a normal healing wound may be used as a reference wound, in which case a suitable test compound for use in the prevention, treatment and/or inhibition of aberrant wound healing may be one that produces a test wound that closely resembles the reference wound.

It will be appreciated that “aberrant wound healing” in the context of the preceding paragraphs encompasses both wound healing that leads to the formation of a chronic wound; and wound healing that leads to the formation of pathological scar, such as a keloid.

The inventors believe that the type of aberrant wound healing response that arises may be determined by the nature of the wound formed on the transgenic animal. For example, an incisional wound or punch biopsy may give rise to aberrant wound healing leading to the formation of a pathological scar. In contrast larger excisional wounds (for example, in the case of a rodent model, a full-thickness wound of approximately 2 cm by 2 cm) may give rise to aberrant wound healing leading to the formation of a chronic wound.

The skilled person will appreciate that a wide range of test compounds may be investigated in accordance with the preceding aspect of the invention, and that suitable test compounds are in no way limited to antagonists of CXCL13 or CXCR5 activity.

In a further aspect of the invention there is provided a method of preventing and/or inhibiting pathological scarring, the method comprising administering a therapeutically effective amount of an antagonist of CXCL13 or CXCR5 activity to a patient in need of such prevention and/or inhibition.

In a still further aspect of the invention there is provided a method of preventing and/or treating a chronic wound, the method comprising administering a therapeutically effective amount of an antagonist of CXCL13 or CXCR5 activity to a patient in need of such prevention and/or treatment.

Antagonists suitable for use in accordance with the methods of treatment of the invention may be selected with reference to any of the preceding considerations.

A therapeutically effective amount of a suitable antagonist may be determined with reference to appropriate considerations outlined elsewhere in the specification.

The present invention also provides a method of treating a chronic wound, to promote healing of said chronic wound, the method comprising:
i) debriding the wound;
ii) assessing expression of CXCL13 or CXCR5 in the skin bordering the debrided tissue;
   and repeating steps i) and ii) until expression of CXCL13 or CXCR5 in the skin bordering the debrided tissue falls below a reference value. Preferably the assessment may be conducted utilising an in vitro method.

The skilled person will appreciate that much the same information may be provided by the assessment of expression of CXCL13 or CXCR5 in debrided tissue itself, and the method defined in the preceding paragraph may be modified to investigate gene expression in the debrided tissue. In certain circumstances this modification may be preferred, since the debrided tissue, being already removed from the body, may be more amenable to ex vivo analysis.

A suitable reference value for use in such methods may be determined having regard to the level of expression of CXCL13 or CXCR5 observed in normal skin. For example, a suitable reference value may be at least 30 times the level of expression found in normal skin, more preferably at least 20 times the level of expression found in normal skin, even more preferably at least 10 times the level of expression found in normal skin, yet more preferably at least 5 times the level of expression found in normal skin, and most preferably less than 5 times the level of expression found in normal skin.

A method of treating a chronic wound in accordance with the preceding aspects of the invention may optionally include the administration of a medicament for the treatment of chronic wounds (such as a medicament suitable for the promotion of wound healing).

The invention also provides a method of assessing the therapeutic effectiveness of a treatment administered to a wound, the method comprising assessing CXCL13 or CXCR5 expression in a wound to which a treatment has been administered, and comparing this expression with a reference value representative of CXCL13 or CXCR5 expression in unwounded skin, wherein increased expression of CXCL13 or CXCR5 in the wound, as compared to the reference value, indicates that the administered treatment is not therapeutically effective. Thus continued elevated expression of CXCL13 or CXCR5 during the administration of a treatment intended to improve healing of the wound provides an indication that the treatment is not therapeutically effective.

The information provided by this method in accordance with the invention may let a clinician responsible for the treatment of a wound know that a treatment that has been utilised has not been therapeutically effective, and that it may therefore be preferable to employ an alternative treatment. Preferably the wound may be a chronic wound, and the treatment administered may be one intended to increase the rate of healing of the chronic wound. Alternatively the wound may be one that is believed to be at heightened risk of pathological scar formation, and the treatment administered may be one intended to prevent pathological scar formation.

Expression of CXCL13 or CXCR5 in materials derived from patients (such as debrided tissue, or skin or wound samples) may be measured at the level of mRNA and/or protein. Suitable analysis may include derived peptide and nucleic acid fragments, splice variants, polymorphic variants and homologous species encoding or having greater than 50% identity, or more preferably greater than 80% identity, to CXCL13 or CXCR5 peptide sequence (excluding all other known CXC chemokines or chemokine receptors). The expression information derived may be used for diagnosis, prognosis and wound and patient management decisions.

CXCL13 or CXCR5 mRNA levels may be measured by methods involving gene-specific hybridisation, including but not limited to nucleic acid arrays, PCR, NASBA, RCA, branched chain nucleic acid and invader assays, aptamers and antibodies or antibody derivatives (Singh et al, 1993; Boeckh and Boivin 1998; Bloom and Dean, 2003; Jain, 2004; Millar and Moore, 2004; Olson, 2004; Yang and Rothman, 2004).

CXCL13 or CXCR5 protein levels may be measured by aptamer detection, mass spectrometry, NMR, Western blotting and other antibody based methods such as ELISA, RIA and other methods well-known to those skilled in the art (Singh et al, 1993; Crowther, 1995; Bloom and Dean, 2003). Furthermore, it is to be appreciated that many of the reagents considered as antagonists of CXCL13 signalling may also be used for the direct or indirect measurement of CXCL13 nucleic acid sequences, proteins and peptides and biological activity.

The skilled person will appreciate that suitable antibodies, antibody derivatives, aptamers and other nucleic acids for use in the assessment of CXCL13 or CXCR5 levels may be selected with reference to the criteria discussed elsewhere in the specification in connection with antagonists of CXCL13 or CXCR5 activity.

The invention will now be described further by way of example with reference to the following experimental methods and results, and with reference to the accompanying Figure in which:

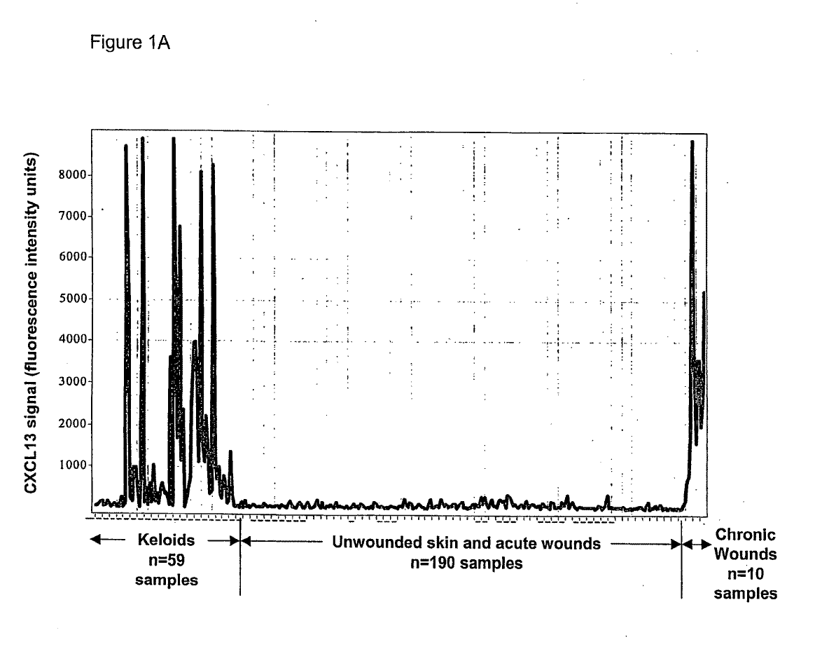

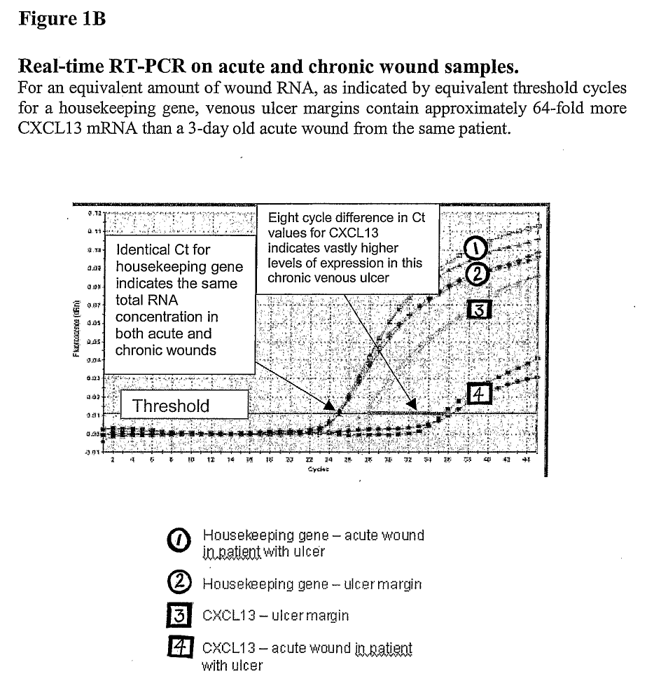

FIG. 1A provides a comparison of CXCL13 expression in a sample of pathological keloid scars, chronic wounds, and unwounded skin; and

FIG. 1B provides a comparison of real-time RT-PCR on acute and chronic wound samples.

The accompanying Sequence Information section sets out the amino acid sequences of CXCL13 and CXCR5, as well as the sequences of cDNA molecules encoding these proteins, and primers and probes used for the quantification of CXCL13 expression.

### EXPERIMENTAL METHODS AND RESULTS

### 1.1 Sources of Human Samples

1.1.1 Normally and Delayed (Chronic) Healing Wounds from White Caucasian Male and Female Patients with Wound Healing Disorders

Subjects: Five male and female patients with clinically diagnosed chronic venous ulcers.

Procedures: Subjects' chronic wounds were monitored for a minimum of 6 weeks prior to day 0. On day 0 each subject received, on the inner aspect of each arm, two full-thickness 4 mm punch biopsy wounds spaced at least 5 cm from each other and one 1 cm linear incision at least 3 cm from the punch biopsies (normally healing wounds). Subsequently, each healing wound/scar was excised at specific time points for gene expression analysis of these acute healing wounds in the arm (day 3 for incision and one punch biopsy, day 30 for the other punch biopsy). 6 mm full-thickness punch biopsies of ulcer edges were taken at the time of the day 3 visit and these chronic wound samples (delayed healing wounds) were also used for gene expression analysis. Excised tissue was immediately frozen in liquid nitrogen and stored at −80° C. until required for RNA extraction.

1.1.2 Healing Wounds from Individuals Susceptible to Keloid Information

Subjects: Jamaican individuals presenting keloid scars that had been established for at least one year and who were to undergo surgical removal of the keloid scar. The age of the scar, twinned with a thorough examination of the scar history, ensured that the scar had been correctly diagnosed as keloidal and not hypertrophic.

Procedures: Two separate full-thickness excisional ellipses were excised from the keloid. The ellipses spanned the margin of the keloid and ran perpendicular to the margin of the keloid. The biopsies were sectioned into three segments comprising the skin bordering the keloid (termed extra-keloid herein), the margin of the keloid (termed peri-keloid) and the keloid tissue itself (termed intra-keloid). The biopsy sections were immersed in RNA Later solution (Ambion) and stored at −80° C. Three or four days post-excision, one of the ellipsoid wounds was re-excised and the biopsy was sectioned and treated as above. Seven or ten days post-excision, the remaining ellipsoid wound was re-excised.

The separation of keloid biopsies into horizontal domains in the manner described above, allows investigation of the molecular differences between keloid-susceptible skin (represented by the extra-keloid portion), the margin of a keloid (the peri-keloid portion) and the interior of a keloid (the intra-keloid portion).

1.1.3 Normally Healing Wounds and Unwounded Skin from Young Healthy White Caucasian Males

Subjects: Sixty non-smoking, healthy, Caucasian, males aged 18 to 45 years.

Procedures: All subjects had two 1 cm linear incisions spaced at least 5 cm from each other on the inner aspect of each arm. A proportion of the subjects also received one 3 mm punch biopsy on each arm (at least 3 cm from the incisions). The 3 mm punch biopsies were reserved so that they could be used as examples of unwounded skin. Each healing wound/scar was excised at certain time points post-wounding for gene expression analysis, including day 1, day 3, day 5, day 30 and day 180. Excised tissue was immediately frozen in liquid nitrogen and stored at −80° C. until required for RNA extraction.

### 1.1.4 Skin Front the Abdomen of Afro-Caribbean Females

Subjects: Jamaican individuals without a history of keloid scarring.

Procedures: Subjects had one centimetre ellipses of abdominal skin excised to a depth of one centimetre prior to abdominoplasty. The biopsy sections were immersed in RNA Later solution (Ambion) and stored at −80° C. prior to gene expression profiling. Three days post-excision, one of the ellipsoid wounds was re-excised and the biopsy was sectioned and treated as described above. Seven days post-excision, the remaining ellipsoid wound was re-excised.

### 1.1.5 RNA From Normal and Inflamed Tonsil Tissue of Females

RNA from normal and inflamed tonsil tissue from human females was obtained from Clinomics Biosciences Inc (Waterviliet, N.Y. 12189).

1.1.6 Normally Healing Wounds and Unwounded Skin from White Caucasian Post-Menopausal Females

Subjects: Nine post-menopausal females not receiving hormone replacement therapy.

Procedures: Four 3 mm punch biopsy wounds were created in the inner aspect of both arms and the biopsies were reserved as unwounded skin samples. The biopsies were re-excised at 3, 7 and 30 days post wounding. Excised tissue was immediately frozen in liquid nitrogen and stored at −80° C. until required for RNA extraction.

### 1.2 Affymetrix GeneChip Expression Profiling

Tissues were disrupted using a Diax (G-10) homogeniser and total RNA was purified using the skin-specific protocol of the RNeasy midikit (Qiagen). Gene expression was determined on HGU133a arrays using standard methods on the Affymetrix GeneChip system (GeneChip Expression Analysis Technical Manual, Affymetrix 701021 rev 1, 2001).

### 1.3 Analysis of GeneChip Expression Data

Data sets passing quality control metrics recommended by Affymetrix (percent present, scaling factor, GAPDH 3′/5′ ratio) were imported into Spotfire (Spotfire AB). Genes were classified on the basis of the Affymetrix p values as being called present in one, two, three etc array data sets. Genes called absent on all arrays were omitted from further analyses.

### 1.4 Real-Time Reverse Transcription Polymerase Chain Reaction

Quantitative real-time reverse transcription PCR (real-time RT PCR) was used to quantify the level of CXCL13 expression relative to a house-keeping gene (5-AMP activated protein kinase gamma 1 subunit). RNA produced was treated to remove contaminating DNA according to manufacturers instructions (DNA-free, Ambion Inc.) and subjected to reverse transcription/polymerase chain reaction in the Stratagene MX4000 thermal cycler according to manufacturers instructions (Stratagene One Step RT-PCR kit: Cat. No 929532). Primers and probes used are shown in the Sequence Information section.

Final composition of reaction mix (25 μl total volume) was as follows: template RNA (˜250 ng), 1× Stratagene Core RT buffer, 0.8 mM dNTPs, 3.5 mM MgCl₂, 75 nM Rox, 1.25 Units Sure Start Taq, 400 nM each primer, 200 nM probe, 10 Units reverse transcriptase.

Cycling conditions used were: 1×30 min at 45° C.; 1×10 min at 95° C.; 45×(30 sec at 95° C., 1 min at 58° C., 30 sec at 72° C.).

Controls with no template and no reverse transcriptase were used to ensure amplification from RNA only.

### 2 Results

### 2.1 Affymetrix Data Indicate that CXCL13 is Expressed in the Margins of Chronic Wounds and Wounds that are Destined to Become Keloids, But Not in Normal Wounds or Unwounded Skin

CXCL13 expression was detected in 9 out of 10 venous ulcer samples from five separate patients and 42 from 59 acute wounds destined to become keloids including 6 from 8 wounds in extra-keloidal, macroscopically normal tissue. By way of contrast, no CXCL13 expression was detected (gene expression below the lower limit of detection) in multiple acute wounds from all five patients with chronic wounds, all six wounds from non-keloid forming Jamaicans and in 218 human acute wounds from males and females at various stages of healing.

The signal representative of CXCL13 expression was 58-fold higher in chronic wounds than in acute wounds from the same patients (p=1.55×10⁻¹⁰ for students t-test on log₂ data) and 33-fold higher than in all the acute human wounds (p=2.90×10⁻²⁵ for students t-test on log₂ data). The skilled person will recognise that these results clearly indicate that CXCL13 expression and activity is associated with the development and/or maintenance of chronic wounds, and that antagonists of CXCL13 activity (or of the activity of CXCL13's receptor, CXCR5) may therefore be used in the prevention and/or treatment of chronic wounds. A reduction in the expression levels of CXCL13 may also correlate with the healing status of chronic wounds. This information may be useful as a diagnostic indicator of treatment efficacy. For example, if CXCL13 expression levels at the margin of a chronic wound remain high after several weeks of treatment, this would indicate to the medical practitioner that the wound was not responding to treatment and that an alternative treatment regimen should be used.

The signal representative of CXCL13 expression was 20-fold higher in acute wounds destined to become keloids than in control samples from acute wounds derived from non-keloid forming Jamaicans (p<1.2×10⁻⁴ for students t-test on log data). The inventors believe that this fold change difference is significantly underestimated; the Affymetrix algorithm defines the signal from all acute wounds as ‘absent’ and thus the signal value from these wounds will be overestimated due to system noise. Certainly the inventors believe that these results are sufficient to serve to illustrate to the skilled person that CXCL13 expression and activity is associated with the development of pathological keloid scarring, and that antagonists of CXCL13 activity (or of the activity of CXCL13's receptor CXCR5) may therefore be used in the prevention and/or inhibition of pathological scars such as keloids. The results of this experiment also clearly indicate that increased expression of CXCL13 or CXCR5 at the site of a wound may be associated with a predisposition of such a wound to keloid formation. Thus, investigation of the level of expression of CXCL13 or CXCR5 at a wound site may be used as a prognostic tool to evaluate the risk that the wound investigated will develop into a pathological keloid scar. Furthermore, the involvement of CXCL13 or CXCR5 activity in fibrosis associated with keloids provides a clear indication that antagonism of these molecules may be therapeutically effective in prevention, reduction or treatment of fibrotic disorders.

### 2.2 Analysis of Control and Inflamed Tonsil Tissue Surprisingly Reveal that CXCL13 Expression is not Associated with Inflammation

In order to investigate whether CXCL13 expression is associated with inflammation, such as that occurring during the initial stages of wound healing, expression of CXCL13 was investigated in both inflamed and normal tonsil tissue. The results of this study showed that CXCL13 expression is not increased in inflamed tissue as opposed to control tonsil, thus indicating that CXCL13 expression is linked to the maintenance of lymphoid tissue rather than inflammation per se.

This finding was further supported by the fact that no expression of CXCL13 was detected in the many samples taken from wounds during the inflammatory phase of healing (1, 3 and 5 days post-wounding).

### 2.3 Real-Time RT-PCR Experiments Corroborate Affymetrix Measurements for CXCL13

Real-time RT-PCR indicates approximately 64-fold higher expression of CXCL13 in chronic wounds as compared to acute wounds (see Table 1). In a real-time RT-PCR experiment the threshold cycle (Ct) indicates the quantity of gene transcript present. A lower Ct value indicates a higher level of target transcript providing that controls run without reverse transcriptase (−rt) produce a much higher Ct value—indicating no or insignificant amplification of the gene sequence from genomic DNA contamination. Our validation experiments demonstrate that for any given total RNA input (as indicated by the Ct for a housekeeping gene) the Ct value for CXCL13 occurs at least 6 cycles earlier in a chronic venous ulcer sample as compared to acute wounds from the same and other patients indicating approximately 2⁶ or 64-fold higher expression (see Table 1). In most instances expression of CXCL13 was undetectable in acute wounds (Ct of 45 cycles, see Table 1).

A further illustration of the results investigating expression of CXCL13 in pathological keloid scars, chronic wounds and acute wounds can be seen in FIG. 1. Here it can be seen that CXCL13 expression is notably increased in pathological keloid scars (and wounds which develop into such scars) and, chronic wounds when compared to expression of CXCL13 in acute wounds (other than those which develop into pathological scars). The inventors believe that observed variations in expression levels between different keloid samples are linked to whether or not the keloid is actively undergoing fibrosis at the time of sampling (i.e. that elevated expression is associated with samples undergoing fibrosis in response to biopsy, or samples tested during growth beyond the boundaries of the initial insult, whilst lower expression is found in keloids sampled at a time when the fibrotic response is relatively reduced).

FIG. 1 also shows that CXCL13 expression is notably increased in chronic wounds. In FIG. 1B, identical Ct for the housekeeping investigated in chronic and acute wound samples indicates that both samples contain the same total RNA concentration. In contrast, the eight cycle difference between Ct values for CXCL13 in chronic wound (venous ulcer) and acute wound samples indicates vastly higher levels of expression of CXCL13 in the chronic wound sample than in the acute wound.

TABLE 1
Real-time RT-PCR on acute and chronic wound samples.

| | | |
|---|---|---|
| | Mean Ct | |
| Sample and | Housekeeping | Mean Ct |
| dilution | gene | CXCL13 |

1 day old acute wound

| | | |
|---|---|---|
| 1/10 | 23.5 | 33.1 |
| 1/150 | 25.6 | 45 |
| 1/1500 | 30.8 | 45 |
| 1/15 −rt | 43.1 | 42.3 |

3 day old acute wound

| | | |
|---|---|---|
| 1/15 | 22.7 | 32.4 |
| 1/150 | 25.5 | 45 |
| 1/1500 | 28.7 | 43.9 |
| 1/15 −rt | 45 | 45 |

3 day old acute wound in CVU patient 1

| | | |
|---|---|---|
| 1/10 | 24.8 | 36 |
| 1/100 | 27.4 | 44.9 |
| 1/1000 | 32.9 | 45 |
| 1/10 −rt | 45 | 45 |

3 day old acute wound in CVU patient 2

| | | |
|---|---|---|
| 1/15 | 24.37 | 45 |
| 1/150 | 27.57 | 45 |
| 1/1500 | 32.12 | 45 |
| 1/15 −rt | 45 | 45 |

3 day old acute wound in CVU patient 2

| | | |
|---|---|---|
| 1/15 | 24.44 | 41.11 |
| 1/150 | 27.7 | 45 |
| 1/1500 | 34.51 | 45 |
| 1/15 −rt | 45 | 45 |

14 day old acute wound

| | | |
|---|---|---|
| 1/15 | 24.94 | 33.82 |
| 1/150 | 28.14 | 45 |
| 1/1500 | 34.53 | 45 |
| 1/15 −rt | 45 | 45 |

Chronic venous ulcer edge, patient 1

| | | |
|---|---|---|
| 1/10 | 25.1 | 27.7 |
| 1/100 | 27.5 | 29.8 |
| 1/1000 | 30.9 | 33 |
| 1/10 −rt | 45 | 45 |

Chronic venous ulcer edge, patient 2 biopsy A

| | | |
|---|---|---|
| 1/15 | 24.92 | 26.69 |
| 1/150 | 28.93 | 28.37 |
| 1/1500 | 40.99 | 33.59 |
| 1/15 −rt | 45 | 45 |

Chronic venous ulcer edge, patient 2 biopsy B

| | | |
|---|---|---|
| 1/15 | 25.13 | 26.99 |
| 1/150 | 28.11 | 28.33 |
| 1/1500 | 33.2 | 34.08 |
| 1/15 −rt | 45 | 45 |

−rt, without reverse transcriptase; CVU, chronic venous ulcer.
A threshold cycle (Ct)of 45 means that the target sequence was undetectable.

### CONCLUSIONS

CXCL13 is not expressed in unwounded skins.

CXCL13 is not expressed in acute, healing wounds that give rise to normal scarring.

CXCL13 expression is not associated with any stage of normal healing.

CXCL13 is expressed in tonsil tissue.

CXCL13 expression is not higher in inflamed as compared to non-inflamed tonsil tissue.

CXCL13 is not associated with inflammation in either wound healing or disease states.

Surprisingly, CXCL13 is highly expressed in the margins of chronic venous ulcers and in wounded tissue destined to form a keloid scar. Thus antagonism of CXCL13 and/or CXCR5 activity represents a suitable therapeutic intervention for the prevention, treatment or reduction of aberrant wound healing conditions such as chronic wounds or keloid scars.

Furthermore, antagonism of CXCL13 or CXCR5 may be of use in the prevention, treatment or reduction of fibrotic disorders.

### SEQUENCE INFORMATION

Nucleic acid and protein sequences of human CXCL13 and CXCR5

Amino acid sequence of human CXCL13 (including leader sequence)

Sequence ID No. 1

| | |
|---|---|
| 1 | mkfistslll mllvsslspv qgvlevyyts lrcrcvqess vfiprrfidr iqilprgngc |
| 61 | prkeiivwkk nksivcvdpq aewiqrmmev lrkrssstlp vpvfkrkip |

Amino acid sequence of human CXCL13 (without leader sequence)

Sequence ID No. 2

| | |
|---|---|
| 1 | vlevyytslr crcvqessvf iprrfidriq ilprgngcpr keiivwkknk sivcvdpqae |
| 61 | wiqrmmevlr krssstlpvp vfkrkip |

Amino acid sequence of CXCR5 isoform 1

Sequence ID No. 3

| | |
|---|---|
| 1 | mnypltlemd lenledlfwe ldrldnyndt slvenhlcpa tegplmasfk avfvpvaysl |
| 61 | ifllgvignv lvlvilerhr qtrsstetfl fhlavadlll vfilpfavae gsvgwvlgtf |
| 121 | lcktvialhk vnfycsslll aciavdryla ivhavbayrh rrllsihitc gtiwlvgfll |
| 181 | alpeilfakv sqghhnnslp rctfaqenqa ethawftsrf lyhvagfllp mlvmgwcyvg |
| 241 | vvhrlrqaqr rpqrqkavrv ailvtsiffl cwspyhivif ldtlarlkav dntcklngsl |
| 301 | pvaitmcefl glahcclnpm lytfagvkfr sdlsrlltkl gctgpaslcq lfpswrrssl |
| 361 | sesenatslt tf |

Amino acid sequence of CXCR5 isoform 2

Sequence ID No. 4

| | |
|---|---|
| 1 | masfkavfvp vayslifllg vignvlvlvi lerhrqtrss tetflfhlav adlllvfilp |
| 61 | favaegsvgw vlgtflcktv ialhkvnfyc sslllaciav drylaivhav hayrhrrlls |
| 121 | ihitcgtiwl vgfllalpei lfakvsqghh nnslprctfs qenqaethaw ftsrflyhva |
| 181 | gfllpmlvmg wcyvgvvhrl rqaqrrpqrq kavrvailvt sifflcwspy hivifldtla |
| 241 | rlkavdntck lngslpvait mceflglahc clnpmlytfa gvkfrsdlsr lltklgctgp |
| 301 | aslcqlfpsw rrsslsesen atslttf |

cDNA encoding human CXCL13

Sequence ID No. 5

| | |
|---|---|
| 1 | ttcggcactt gggagaagat gtttgaaaaa actgactctg ctaatgagcc tggactcaga |
| 61 | gctcaagtct gaactctacc tccagacaga atgaagttca tctcgacatc tctgcttctc |
| 121 | atgctgctgg tcagcagcct ctctccagtc caaggtgttc tggaggtcta ttacacaagc |
| 181 | ttgaggtgta gatgtgtcca agagagctca gtctttatcc ctagacgctt cattgatcga |
| 241 | attcaaatct tgccccgtgg gaatggttgt ccaagaaaag aaatcatagt ctggaagaag |
| 301 | aacaagtcaa ttgtgtgtgt ggaccctcaa gctgaatgga tacaaagaat gatggaagta |
| 361 | ttgagaaaaa gaagttcttc aactctacca gttccagtgt ttaagagaaa gattccctga |
| 421 | tgctgatatt tccactaaga acacctgcat tcttccctta tccctgctct ggattttagt |
| 481 | tttgtgctta gttaaatctt ttccagggag aaagaacttc cccatacaaa taaggcatga |
| 541 | ggactatgtg aaaaataacc ttgcaggagc tgatggggca aactcaagct tcttcactca |
| 601 | cagcacccta tatacacttg gagtttgcat tcttattcat cagggaggaa agtttctttg |
| 661 | aaaatagtta ttcagttata agtaatacag gattattttg attatatact tgttgtttaa |
| 721 | tgtttaaaat ttcttagaaa acaatggaat gagaatttaa gcctcaaatt tgaacatgtg |
| 781 | gcttgaatta agaagaaaat tatggcatat attaaaagca ggcttctatg aaagactcaa |
| 841 | aaagctgcct gggaggcaga tggaacttga gcctgtcaag aggcaaagga atccatgtag |
| 901 | tagatatcct ctgcttaaaa actcactacg gaggagaatt aagtcctact tttaaagaat |
| 961 | ttctttataa aatttactgt ctaagattaa tagcattcga agatccccag acttcataga |
| 1021 | atactcaggg aaagcattta aagggtgatg tacacatgta tcctttcaca catttgcctt |
| 1081 | gacaaacttc tttcactcac atctttttca ctgacttttt ttgtgggggc ggggccgggg |
| 1141 | ggactctggt atctaattct ttaatgattc ctataaatct aatgacattc aataaagttg |
| 1201 | agcaaacatt ttactt |

CDNA encoding human CXCR5 transcript variant 1

Sequence ID No. 6

| | |
|---|---|
| 1 | gctgccacct ctctagaggc acctggcggg gagcctctca acataagaca gtgaccagtc |
| 61 | tggtgactca cagccggcac agccatgaac tacccgctaa cgctggaaat ggacctcgag |
| 121 | aacctggagg acctgttctg ggaactggac agattggaca actataacga cacctccctg |
| 181 | gtggaaaatc atctctgccc tgccacagag gggcccctca tggcctcctt caaggccgtg |
| 241 | ttcgtgcccg tggcctacag cctcatcttc ctcctgggcg tgatcggcaa cgtcctggtg |
| 301 | ctggtgatcc tggagcggca ccggcagaca cgcagttcca cggagacctt cctgttccac |
| 361 | ctggccgtgg ccgacctcct gctggtcttc atcttgccct ttgccgtggc cgagggctct |
| 421 | gtgggctggg tcctggggac cttcctctgc aaaactgtga ttgccctgca caaagtcaac |
| 481 | ttctactgca gcagcctgct cctggcctgc atcgccgtgg accgctacct ggccattgtc |
| 541 | cacgccgtcc atgcctaccg ccaccgccgc ctcctctcca tccacatcac ctgtgggacc |
| 601 | atctggctgg tgggcttcct ccttgccttg ccagagattc tcttcgccaa agtcagccaa |
| 661 | ggccatcaca acaactccct gccacgttgc accttctccc aagagaacca agcagaaacg |
| 721 | catgcctggt tcacctcccg attcctctac catgtggcgg gattcctgct gcccatgctg |
| 781 | gtgatgggct ggtgctacgt gggggtagtg cacaggttgc gccaggccca gcggcgccct |
| 841 | cagcggcaga aggcagtcag ggtggccatc ctggtgacaa gcatcttctt cctctgctgg |
| 901 | tcaccctacc acatcgtcat cttcctggac accctggcga ggctgaaggc cgtggacaat |
| 961 | acctgcaagc tgaatggctc tctccccgtg gccatcacca tgtgtgagtt cctgggcctg |
| 1021 | gcccactgct gcctcaaccc catgctctac actttcgccg gcgtgaagtt ccgcagtgac |
| 1081 | ctgtcgcggc tcctgacgaa gctgggctgt accggccctg cctccctgtg ccagctcttc |
| 1141 | cctagctggc gcaggagcag tctctctgag tcagagaatg ccacctctct caccacgttc |
| 1201 | taggtcccag tgtccccttt tattgctgct tttccttggg gcaggcagtg atgctggatg |
| 1261 | ctccttccaa caggagctgg gatcctaagg gctcaccgtg gctaagagtg tcctaggagt |
| 1321 | atcctcattt ggggtagcta gaggaaccaa cccccatttc tagaacatcc ctgccagctc |
| 1381 | ttctgccggc cctggggcta ggctggagcc cagggagcgg aaagcagctc aaaggcacag |
| 1441 | tgaaggctgt ccttacccat ctgcaccccc ctgggctgag agaacctcac gcacctccca |
| 1501 | tcctaatcat ccaatgctca agaaacaact tctacttctg cccttgccaa cggagagcgc |
| 1561 | ctgcccctcc cagaacacac tccatcagct taggggctgc tgacctccac agcttcccct |
| 1621 | ctctcctcct gcccacctgt caaacaaagc cagaagctga gcaccagggg atgagtggag |
| 1681 | gttaaggctg aggaaaggcc agctggcagc agagtgtggc cttcggacaa ctcagtccct |
| 1741 | aaaaacacag acattctgcc aggcccccaa gcctgcagtc atcttgacca agcaggaagc |
| 1801 | tcagactggt tgagttcagg tagctgcccc tggctctgac cgaaacagcg ctgggtccac |
| 1861 | cccatgtcac cggatcctgg gtggtctgca ggcagggctg actctaggtg cccttggagg |
| 1921 | ccagccagtg acctgaggaa gcgtgaaggc cgagaagcaa gaaagaaacc cgacagaggg |
| 1981 | aagaaaagag ctttcttccc gaaccccaag gagggagatg gatcaatcaa acccggcggt |
| 2041 | cccctccgcc aggcgagatg gggtggggtg gagaactcct agggtggctg ggtccagggg |
| 2101 | atgggaggtt gtgggcattg atggggaagg aggctggctt gtcccctcct cactcccttc |
| 2161 | ccataagcta tagacccgag gaaactcaga gtcggaacgg agaaaggtgg actggaaggg |
| 2221 | gcccgtggga gtcatctcaa ccatcccctc cgtggcatca ccttaggcag ggaagtgtaa |
| 2281 | gaaacacact gaggcaggga agtccccagg ccccaggaag ccgtgccctg cccccgtgag |
| 2341 | gatgtcactc agatggaacc gcaggaagct gctccgtgct tgtttgctca cctggggtgt |
| 2401 | gggaggcccg tccggcagtt ctgggtgctc cctaccacct ccccagcctt tgatcaggtg |
| 2461 | gggagtcagg gacccctgcc cttgtcccac tcaagccaag cagccaagct ccttgggagg |
| 2521 | ccccactggg gaaataacag ctgtggctca cgtgagagtg tcttcacggc aggacaacga |
| 2581 | ggaagcccta agacgtccct tttttctctg agtatctcct cgcaagctgg gtaatcgatg |
| 2641 | ggggagtctg aagcagatgc aaagaggcaa gaggctggat tttgaatttt ctttttaata |
| 2701 | aaaaggcacc tataaaacag gtcaatacag tacaggcagc acagagaccc ccggaacaag |
| 2761 | cctaaaaatt gtttcaaaat aaaaaccaag aagatgtctt caaaaaaaaa aaaaaaaaaa |
| 2821 | aaaa |

CDNA encoding human CXCR5 transcript variant 2

Sequence ID No. 7

| | |
|---|---|
| 1 | ccactctaag gaatgcggtc cctttgacag gcgaaaaact gaagttggaa aagacaaagt |
| 61 | gatttgttca aaattgaaat ttgaaacttg acatttggtc agtgggccct atgtaggaaa |
| 121 | aaacctccaa gagagctagg gttcctctca gagaggaaag acaggtcctt aggtcctcac |
| 181 | cctcccgtct ccttgccctt gcagttctgg gaactggaca gattggacaa ctataacgac |
| 241 | acctccctgg tggaaaatca tctctgccct gccacagagg ggcccctcat ggcctccttc |
| 301 | aaggccgtgt tcgtgcccgt ggcctacagc ctcatcttcc tcctgggcgt gatcggcaac |
| 361 | gtcctggtgc tggtgatcct ggagcggcac cggcagacac gcagttccac ggagaccttc |
| 421 | ctgttccacc tggccgtggc cgacctcctg ctggtcttca tcttgccctt tgccgtggcc |
| 481 | gagggctctg tgggctgggt cctggggacc ttcctctgca aaactgtgat tgccctgcac |
| 541 | aaagtcaact tctactgcag cagcctgctc ctggcctgca tcgccgtgga ccgctacctg |
| 601 | gccattgtcc acgccgtcca tgcctaccgc caccgccgcc tcctctccat ccacatcacc |
| 661 | tgtgggacca tctggctggt gggcttcctc cttgccttgc cagagattct cttcgccaaa |
| 721 | gtcagccaag gccatcacaa caactccctg ccacgttgca ccttctccca agagaaccaa |
| 781 | gcagaaacgc atgcctggtt cacctcccga ttcctctacc atgtggcggg attcctgctg |
| 841 | cccatgctgg tgatgggctg gtgctacgtg ggggtagtgc acaggttgcg coaggcccag |
| 901 | cggcgccctc agcggcagaa ggcagtcagg gtggccatcc tggtgacaag catcttcttc |
| 961 | ctctgctggt caccctacca catcgtcatc ttcctggaca ccctggcgag gctgaaggcc |
| 1021 | gtggacaata cctgcaagct gaatggctct ctccccgtgg ccatcaccat gtgtgagttc |
| 1081 | ctgggcctgg cccactgctg cctcaacccc atgctctaca ctttcgccgg cgtgaagttc |
| 1141 | cgcagtgacc tgtcgcggct cctgacgaag ctgggctgta ccggccctgc ctccctgtgc |
| 1201 | cagctcttcc ctagctggcg caggagcagt ctctctgagt cagagaatgc cacctctctc |
| 1261 | accacgttct aggtcccagt gtcccctttt attgctgctt ttccttgggg caggcagtga |
| 1321 | tgctggatgc tccttccaac aggagctggg atcctaaggg ctcaccgtgg ctaagagtgt |
| 1381 | cctaggagta tcctcatttg gggtagctag aggaaccaac ccccatttct agaacatccc |
| 1441 | tgccagctct tctgccggcc ctggggctag gctggagccc agggagcgga aagcagctca |
| 1501 | aaggcacagt gaaggctgtc cttacccatc tgcacccccc tgggctgaga gaacctcacg |
| 1561 | cacctcccat cctaatcatc caatgctcaa gaaacaactt ctacttctgc ccttgccaac |
| 1621 | ggagagcgcc tgcccctccc agaacacact ccatcagctt aggggctgct gacctccaca |
| 1681 | gcttcccctc tctcctcctg cccacctgtc aaacaaagcc agaagctgag caccagggga |
| 1741 | tgagtggagg ttaaggctga ggaaaggcca gctggcagca gagtgtggcc ttcggacaac |
| 1801 | tcagtcccta aaaacacaga cattctgcca ggcccccaag cctgcagtca tcttgaccaa |
| 1861 | gcaggaagct cagactggtt gagttcaggt agctgcccct ggctctgacc gaaacagcgc |
| 1921 | tgggtccacc ccatgtcacc ggatcctggg tggtctgcag gcagggctga ctctaggtgc |
| 1981 | ccttggaggc cagccagtga cctgaggaag cgtgaaggcc gagaagcaag aaagaaaccc |
| 2041 | gacagaggga agaaaagagc tttcttcccg aaccccaagg agggagatgg atcaatcaaa |
| 2101 | cccggcggtc ccctccgcca ggcgagatgg ggtggggtgg agaactccta gggtggctgg |
| 2161 | gtccagggga tgggaggttg tgggcattga tggggaagga ggctggcttg tcccctcctc |
| 2221 | actcccttcc cataagctat agacccgagg aaactcagag tcggaacgga gaaaggtgga |
| 2281 | ctggaagggg cccgtgggag tcatctcaac catcccctcc gtggcatcac cttaggcagg |
| 2341 | gaagtgtaag aaacacactg aggcagggaa gtccccaggc cccaggaagc cgtgccctgc |
| 2401 | ccccgtgagg atgtcactca gatggaaccg caggaagctg ctccgtgctt gtttgctcac |
| 2461 | ctggggtgtg ggaggcccgt ccggcagttc tgggtgctcc ctaccacctc cccagccttt |
| 2521 | gatcaggtgg ggagtcaggg acccctgccc ttgtcccact caagccaagc agccaagctc |
| 2581 | cttgggaggc cccactgggg aaataacagc tgtggctcac gtgagagtgt cttcacggca |
| 2641 | ggacaacgag gaagccctaa gacgtccctt ttttctctga gtatctcctc gcaagctggg |
| 2701 | taatcgatgg gggagtctga agcagatgca aagaggcaag aggctggatt ttgaattttc |
| 2761 | tttttaataa aaaggcacct ataaaacagg tcaatacagt acaggcagca cagagacccc |
| 2821 | cggaacaagc ctaaaaattg tttcaaaata aaaaccaaga agatgtcttc aaaaaaaaaa |
| 2881 | aaaaaaaaaa aaa |

Primers and probes used for the quantification of
CXCL13 expression.

CXCL13 (MWG Biotech)

| | |
|---|---|
| R | 5′ tca gct cct gca agg tta tt 3′ |
| L | 5′ tct tcc ctt atc cct gct ct 3′ |
| Probe | 5′ *tcc agg gag aaa gaa ctt ccc ca # 3′ |

Housekeeping gene (Oswel)

| | |
|---|---|
| R | 5′ agc tca ggg ctt ctt ctc tc 3′ |
| L | 5′ ggt tca ccg act tgt agt gg 3′ |
| Probe | 5′ *aca tcc tgc agg ccc tgg tg @ 3′ |

Key
*= 6 FAM
# = Blackhole quencher 1
@ = TAMRA

### REFERENCES

Ansel K M, Ngo V N, Hyman P L, Luther S A, Forster R, Sedgwick J D, Browning J L, Lipp M, Cyster J G (2000). A chemokine-driven positive feedback loop organizes lymphoid follicles. Nature 406, 309-14.
Bloom J C and Dean R A (2003) in Biomarkers in Clinical Drug Development.
Boeckh M, Boivin G (1998). Quantitation of cytomegalovirus: methodologic aspects and clinical applications. Clin. Microbiol. Rev. 11, 533-54.
Crowther J R (1995) in Elisa: Theory and Practice. Humana Press.
Jain K K (2004). Applications of biochips: from diagnostics to personalized medicine. Curr. Opin Drug. Discovery Devel. 7, 285-9.
Kesavadev J D, Short K R, Nair, K S (2003). Diabetes in old age: an emerging epidermic. J. Assoc. Physicians India. 51, 1083-94.
Kurreck J (2003). Antisense technologies. Improvement through novel chemical modifications. Eur. J. Biochem. 270, 1628-44.
Maessen-Visch M B, Hamulyak K, Tazelaar D J, Crombag N H, Neumann H A (1999). The prevalence of factor V Leiden mutation in patients with leg ulcers and venous insufficiency. Arch. Dermatol. 135, 41-4.
Margolis D J, Knauss J, Bilker W (2002). Hormone replacement therapy and prevention of pressure ulcers and venous leg ulcers. Lancet 359, 675-7.
Millar B C, Moore J E (2004). Molecular diagnostics: current options. Methods Mol. Biol. 266, 139-66.
Montgomery M K (2004). RNA interference: historical overview and significance. Methods Mol. Biol. 265, 3-21.
Olson M C, Takova T, Chehak L, Curtis M L, Olson S M, Kwiatkowski R W (2004). Invader assay for RNA quantitation. Methods Mol. Biol. 258, 53-69.
Schutze N (2004). siRNA technology. Mol. Cell. Endocrinol. 213, 115-9.
Singh, P, Shaima, B P, and Typle, P (editors), 1993. Diagnostics in the Year 2000: Antibody, Biosensor, and Nucleic Acid Technologies. (Van Nostrand Reinhold, New York, 1993).
Yang S, Rothman R E (2004). PCR-based diagnostics for infectious diseases: uses, limitations, and future applications in acute-care settings. Lancet Infect. Dis. 4, 337-48

1. The use of an antagonist of CXCL13 or CXCR5 activity in the preparation of a medicament for the prevention and/or inhibition of pathological scarring.

2. The use according to claim 1, wherein the pathological scarring is selected from the group consisting of keloid scarring; hypertrophic scarring; and pterygium.

3. The use according to claim 2, wherein the pathological scarring is keloid scarring.

4. The use of an antagonist of CXCL13 or CXCR5 activity in the preparation of a medicament for the prevention and/or inhibition of a fibrotic disorder.

5. The use according to claim 4, wherein the fibrotic disorder is selected from the group consisting of cirrhosis of the liver; liver fibrosis; glomerulonephritis; conjunctival cicatrisation; pulmonary fibrosis; lung fibrosis; scleroderma; skin fibrosis; muscle fibrosis; radiation fibrosis; kidney fibrosis; renal fibrosis; glomerulosclerosis; uterine fibrosis; fibrosis associated with endometriosis; adhesions; chronic obstructive pulmonary disease; myocardial fibrosis; fibrosis following myocardial infarction; progressive systemic fibrosis; restenosis; fibrosis associated with ischemic disease; central nervous system fibrosis following stroke; fibrosis associated with neuro-degenerative disorders such as multiple sclerosis; and fibrosis associated with proliferative vitreoretinopathy.

6. The use of an antagonist of CXCL13 or CXCR5 activity in the preparation of a medicament for the prevention and/or treatment of a chronic wound.

7. The use according to claim 6, wherein the chronic wound is selected from the group consisting of venous ulcers; ischemic wounds; diabetic ulcers; neurotrophic ulcers; arterial ulcers; wounds infected with microorganisms; wounds of patients with impaired circulation or venous stasis; and decubitus ulcers (pressure sores).

8. The use according to claim 7, wherein the chronic wound is a venous ulcer.

9. The use according to claim 1, wherein the antagonist of CXCL13 or CXCR5 activity is a compound that prevents binding of CXCL13 to CXCR5.

10. The use according to claim 9, wherein the antagonist of CXCL13 is a function neutralising antibody.

11. The use according to claim 9 wherein the compound preventing binding of CXCL13 to CXCR5 is an antibody, a peptide, a soluble form of the CXCR5 receptor or a small molecule antagonist.

12. The use according to claim 10, wherein the function neutralising antibody is selected from the group consisting of MAB801 and MAB190.

13. The use according to claim 1, wherein the antagonist of CXCL13 or CXCR5 activity is a compound that reduces expression of CXCL13 or CXCR5.

14. The use according to claim 12, wherein the compound is selected from the group consisting of antisense nucleic acids, ribozymes, siRNA, aptamers, and LTβR-Ig.

15. The use according to claim 1, wherein the antagonist of CXCL13 or CXCR5 is a compound that inhibits intracellular signalling generated on binding of CXCL13 to CXCR5.

16. The use according to claim 1, wherein the antagonist of CXCL13 or CXCR5 is the product of the MHV-68 M3 gene.

17. The use of a transgenic animal that over-expresses CXCL13 in a model of aberrant wound healing.

18. The use according to claim 17, wherein the aberrant wound healing leads to the formation of a chronic wound.

19. The use according to claim 17, wherein the aberrant wound healing leads to the formation of a pathological scar.

20. A method of screening a test compound for use in the prevention, treatment or inhibition of aberrant wound healing, the method comprising administering the test compound to a dermal wound site of a transgenic animal that over-expresses CXCL13.

21. A method according to claim 20, wherein the aberrant wound healing leads to the formation of a chronic wound.

22. A method according to claim 20, wherein the aberrant wound healing leads to the formation of pathological scar.


SEQUENCE LISTING (13 sequences)

SEQ ID NO: 1 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 2 - protein, 87 aa, Homo sapiens
VLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 3 - protein, 372 aa, Homo sapiens
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 4 - protein, 327 aa, Homo sapiens
MASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 5 - DNA, 1216 bp, Homo sapiens
ttcggcacttgggagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccagggagaaagaacttccccatacaaataaggcatgaggactatgtgaaaaataaccttgcaggagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtgggggcggggccggggggactctggtatctaattctttaatgattcctataaatctaatgacattcaataaagttgagcaaacattttactt

SEQ ID NO: 6 - DNA, 2824 bp, Homo sapiens
gctgccacctctctagaggcacctggcggggagcctctcaacataagacagtgaccagtctggtgactcacagccggcacagccatgaactacccgctaacgctggaaatggacctcgagaacctggaggacctgttctgggaactggacagattggacaactataacgacacctccctggtggaaaatcatctctgccctgccacagaggggcccctcatggcctccttcaaggccgtgttcgtgcccgtggcctacagcctcatcttcctcctgggcgtgatcggcaacgtcctggtgctggtgatcctggagcggcaccggcagacacgcagttccacggagaccttcctgttccacctggccgtggccgacctcctgctggtcttcatcttgccctttgccgtggccgagggctctgtgggctgggtcctggggaccttcctctgcaaaactgtgattgccctgcacaaagtcaacttctactgcagcagcctgctcctggcctgcatcgccgtggaccgctacctggccattgtccacgccgtccatgcctaccgccaccgccgcctcctctccatccacatcacctgtgggaccatctggctggtgggcttcctccttgccttgccagagattctcttcgccaaagtcagccaaggccatcacaacaactccctgccacgttgcaccttctcccaagagaaccaagcagaaacgcatgcctggttcacctcccgattcctctaccatgtggcgggattcctgctgcccatgctggtgatgggctggtgctacgtgggggtagtgcacaggttgcgccaggcccagcggcgccctcagcggcagaaggcagtcagggtggccatcctggtgacaagcatcttcttcctctgctggtcaccctaccacatcgtcatcttcctggacaccctggcgaggctgaaggccgtggacaatacctgcaagctgaatggctctctccccgtggccatcaccatgtgtgagttcctgggcctggcccactgctgcctcaaccccatgctctacactttcgccggcgtgaagttccgcagtgacctgtcgcggctcctgacgaagctgggctgtaccggccctgcctccctgtgccagctcttccctagctggcgcaggagcagtctctctgagtcagagaatgccacctctctcaccacgttctaggtcccagtgtccccttttattgctgcttttccttggggcaggcagtgatgctggatgctccttccaacaggagctgggatcctaagggctcaccgtggctaagagtgtcctaggagtatcctcatttggggtagctagaggaaccaacccccatttctagaacatccctgccagctcttctgccggccctggggctaggctggagcccagggagcggaaagcagctcaaaggcacagtgaaggctgtccttacccatctgcacccccctgggctgagagaacctcacgcacctcccatcctaatcatccaatgctcaagaaacaacttctacttctgcccttgccaacggagagcgcctgcccctcccagaacacactccatcagcttaggggctgctgacctccacagcttcccctctctcctcctgcccacctgtcaaacaaagccagaagctgagcaccaggggatgagtggaggttaaggctgaggaaaggccagctggcagcagagtgtggccttcggacaactcagtccctaaaaacacagacattctgccaggcccccaagcctgcagtcatcttgaccaagcaggaagctcagactggttgagttcaggtagctgcccctggctctgaccgaaacagcgctgggtccaccccatgtcaccggatcctgggtggtctgcaggcagggctgactctaggtgcccttggaggccagccagtgacctgaggaagcgtgaaggccgagaagcaagaaagaaacccgacagagggaagaaaagagctttcttcccgaaccccaaggagggagatggatcaatcaaacccggcggtcccctccgccaggcgagatggggtggggtggagaactcctagggtggctgggtccaggggatgggaggttgtgggcattgatggggaaggaggctggcttgtcccctcctcactcccttcccataagctatagacccgaggaaactcagagtcggaacggagaaaggtggactggaaggggcccgtgggagtcatctcaaccatcccctccgtggcatcaccttaggcagggaagtgtaagaaacacactgaggcagggaagtccccaggccccaggaagccgtgccctgcccccgtgaggatgtcactcagatggaaccgcaggaagctgctccgtgcttgtttgctcacctggggtgtgggaggcccgtccggcagttctgggtgctccctaccacctccccagcctttgatcaggtggggagtcagggacccctgcccttgtcccactcaagccaagcagccaagctccttgggaggccccactggggaaataacagctgtggctcacgtgagagtgtcttcacggcaggacaacgaggaagccctaagacgtcccttttttctctgagtatctcctcgcaagctgggtaatcgatgggggagtctgaagcagatgcaaagaggcaagaggctggattttgaattttctttttaataaaaaggcacctataaaacaggtcaatacagtacaggcagcacagagacccccggaacaagcctaaaaattgtttcaaaataaaaaccaagaagatgtcttcaaaaaaaaaaaaaaaaaaaaaaa

SEQ ID NO: 7 - DNA, 2893 bp, Homo sapiens
ccactctaaggaatgcggtccctttgacaggcgaaaaactgaagttggaaaagacaaagtgatttgttcaaaattgaaatttgaaacttgacatttggtcagtgggccctatgtaggaaaaaacctccaagagagctagggttcctctcagagaggaaagacaggtccttaggtcctcaccctcccgtctccttgcccttgcagttctgggaactggacagattggacaactataacgacacctccctggtggaaaatcatctctgccctgccacagaggggcccctcatggcctccttcaaggccgtgttcgtgcccgtggcctacagcctcatcttcctcctgggcgtgatcggcaacgtcctggtgctggtgatcctggagcggcaccggcagacacgcagttccacggagaccttcctgttccacctggccgtggccgacctcctgctggtcttcatcttgccctttgccgtggccgagggctctgtgggctgggtcctggggaccttcctctgcaaaactgtgattgccctgcacaaagtcaacttctactgcagcagcctgctcctggcctgcatcgccgtggaccgctacctggccattgtccacgccgtccatgcctaccgccaccgccgcctcctctccatccacatcacctgtgggaccatctggctggtgggcttcctccttgccttgccagagattctcttcgccaaagtcagccaaggccatcacaacaactccctgccacgttgcaccttctcccaagagaaccaagcagaaacgcatgcctggttcacctcccgattcctctaccatgtggcgggattcctgctgcccatgctggtgatgggctggtgctacgtgggggtagtgcacaggttgcgccaggcccagcggcgccctcagcggcagaaggcagtcagggtggccatcctggtgacaagcatcttcttcctctgctggtcaccctaccacatcgtcatcttcctggacaccctggcgaggctgaaggccgtggacaatacctgcaagctgaatggctctctccccgtggccatcaccatgtgtgagttcctgggcctggcccactgctgcctcaaccccatgctctacactttcgccggcgtgaagttccgcagtgacctgtcgcggctcctgacgaagctgggctgtaccggccctgcctccctgtgccagctcttccctagctggcgcaggagcagtctctctgagtcagagaatgccacctctctcaccacgttctaggtcccagtgtccccttttattgctgcttttccttggggcaggcagtgatgctggatgctccttccaacaggagctgggatcctaagggctcaccgtggctaagagtgtcctaggagtatcctcatttggggtagctagaggaaccaacccccatttctagaacatccctgccagctcttctgccggccctggggctaggctggagcccagggagcggaaagcagctcaaaggcacagtgaaggctgtccttacccatctgcacccccctgggctgagagaacctcacgcacctcccatcctaatcatccaatgctcaagaaacaacttctacttctgcccttgccaacggagagcgcctgcccctcccagaacacactccatcagcttaggggctgctgacctccacagcttcccctctctcctcctgcccacctgtcaaacaaagccagaagctgagcaccaggggatgagtggaggttaaggctgaggaaaggccagctggcagcagagtgtggccttcggacaactcagtccctaaaaacacagacattctgccaggcccccaagcctgcagtcatcttgaccaagcaggaagctcagactggttgagttcaggtagctgcccctggctctgaccgaaacagcgctgggtccaccccatgtcaccggatcctgggtggtctgcaggcagggctgactctaggtgcccttggaggccagccagtgacctgaggaagcgtgaaggccgagaagcaagaaagaaacccgacagagggaagaaaagagctttcttcccgaaccccaaggagggagatggatcaatcaaacccggcggtcccctccgccaggcgagatggggtggggtggagaactcctagggtggctgggtccaggggatgggaggttgtgggcattgatggggaaggaggctggcttgtcccctcctcactcccttcccataagctatagacccgaggaaactcagagtcggaacggagaaaggtggactggaaggggcccgtgggagtcatctcaaccatcccctccgtggcatcaccttaggcagggaagtgtaagaaacacactgaggcagggaagtccccaggccccaggaagccgtgccctgcccccgtgaggatgtcactcagatggaaccgcaggaagctgctccgtgcttgtttgctcacctggggtgtgggaggcccgtccggcagttctgggtgctccctaccacctccccagcctttgatcaggtggggagtcagggacccctgcccttgtcccactcaagccaagcagccaagctccttgggaggccccactggggaaataacagctgtggctcacgtgagagtgtcttcacggcaggacaacgaggaagccctaagacgtcccttttttctctgagtatctcctcgcaagctgggtaatcgatgggggagtctgaagcagatgcaaagaggcaagaggctggattttgaattttctttttaataaaaaggcacctataaaacaggtcaatacagtacaggcagcacagagacccccggaacaagcctaaaaattgtttcaaaataaaaaccaagaagatgtcttcaaaaaaaaaaaaaaaaaaaaaaa

SEQ ID NO: 8 - DNA, 20 bp, Artificial
  Primer for CXCL13
tcagctcctgcaaggttatt

SEQ ID NO: 9 - DNA, 20 bp, Artificial
  Primer for CXCL13
tcttcccttatccctgctct

SEQ ID NO: 10 - DNA, 23 bp, Artificial
  Probe for CXCL13
tccagggagaaagaacttcccca

SEQ ID NO: 11 - DNA, 20 bp, Artificial
  Primer for housekeeping gene
agctcagggcttcttctctc

SEQ ID NO: 12 - DNA, 20 bp, Artificial
  Primer for housekeeping gene
ggttcaccgacttgtagtgg

SEQ ID NO: 13 - DNA, 20 bp, Artificial
  Probe for housekeeping gene
acatcctgcaggccctggtg